(19) United States
(12) Patent Application Publication
(10) Pub. No.: US 2026/0191428 A1
(43) Pub. Date: Jul. 9, 2026

(54) BREATH COLLECTION TOOL

(71) Applicant: University of Louisville Research Foundation, Inc., Louisville, KY (US)

(72) Inventors: Xiao-an Fu, Louisville, KY (US); Michael Nantz, Louisville, KY (US); Jiapeng Huang, Louisville, KY (US)

(21) Appl. No.: 19/133,843

(22) PCT Filed: Dec. 20, 2023
(86) PCT No.: PCT/US23/85017

§ 371 Date: May 29, 2025

Related U.S. Application Data
(60) Provisional application No. 63/476,503, filed on Dec. 21, 2022.
(60) Provisional application No. 63/581,396, filed on Sep. 8, 2023.

(51) Int. Cl. A61B 5/097; G01N 33/53

(52) U.S. Cl. CPC A61B 5/097; G01N 33/5308; G01N 2800/26

(57) ABSTRACT

A tool for collection of exhaled breath includes a microreactor for capturing and concentrating target compounds in a gaseous sample, such as exhaled breath. An airflow of exhaled breath enters the interior of the microreactor, then contacts the central area of the microreactor and is redirected radially outwards, passing through microfluidic channels formed between micropillars, and exits the microreactor through a plurality of vent holes at the periphery. The micropillars are coated with a capture material, such as a reactive compound capable of forming adducts with carbonyl-containing VOCs. The captured target compounds may then be eluted from the micropillars and analyzed for detection of disease states or other purposes.

This application claims the benefit of U.S. provisional patent application Ser. No. 63/581,396, filed 9 Sep. 2023 for BREATH COLLECTION TOOL and U.S. provisional patent application Ser. No. 63/476,503, filed 21 Dec. 2022 for BREATH COLLECTION TOOL, each of which are incorporated herein by reference.

### GOVERNMENT SUPPORT

This invention was made with government support under grant number 1U18TR003787 awarded by the National Institutes of Health. The government has certain rights in the invention.

### TECHNICAL FIELD

A tool for collection of exhaled breath includes a microreactor for capturing and concentrating target compounds in a gaseous sample, such as exhaled breath. An airflow of exhaled breath enters the interior of the microreactor, then contacts the central area of the microreactor and is redirected radially outwards, passing through microfluidic channels formed between micropillars, and exits the microreactor through a plurality of vent holes at the periphery. The micropillars are coated with a capture material, such as a reactive compound capable of forming adducts with carbonyl-containing VOCs. The captured target compounds may then be eluted from the micropillars and analyzed for detection of disease states or other purposes.

### BACKGROUND OF THE INVENTION

In the United States, as of the end of August 2022, there were more than six million people infected with SARS-CoV-2. More than 190K of these COVID-19 infected patients have died from this pandemic to date. Current FDA-authorized DNA sequencing and reverse transcription-polymerase chain reaction (RT-PCR) diagnostic tests for detection of SARS-CoV-2 have been critical for slowing the spread of the virus and preventing future outbreaks. However, the tests are not practical for field use. These standard diagnostic tests require expensive reagents and must be performed in laboratories certified for handling virulent samples. The diagnostic tests are cumbersome and often take days to complete. The dismal situation of this pandemic is partly caused by the lack of effective screening tools for the pathogen SARS-CoV-2 virus. Particularly, asymptomatic and pre-symptomatic patients are highly contiguous and could have contact with uninfected people before being identified for isolation. Therefore, there is an urgent need for an affordable screening test that is non-invasive, portable and can rapidly detect COVID-19 for timely isolation of infected patients and effective contact tracing of potential SARS-CoV-2 infected cases.

There are a few recently developed rapid COVID-19 detection methods involving the direct detection of SARS-CoV-2 virus proteins using immunoassays. One is to immobilize an antibody of SARS-CoV-2 virus spike protein on graphene-based field-effect-transistor biosensors to bind and detect SARS-CoV-2 virus. Detection of the nucleocapsid protein (NP) antigen of SARS-CoV-2 using immunochromatographic assay might be an effective strategy for the early screening of suspected SARS-CoV-2 patients. Body fluid antibody detection is another effective strategy for the identification of coronavirus infection using blood antibody assays for SARS-CoV-2 spike protein, NP or combination of spike protein and NP. However, these new methods have low detection sensitivity for SARS-CoV-2 RNA for pre-symptomatic and asymptomatic patients.

While SARS-CoV-2 is known to cause substantial pulmonary disease, including pneumonia and acute respiratory distress syndrome, clinicians have observed many extrapulmonary manifestations of COVID-19. COVID-19 exhibited cardiac manifestations including stress cardiomyopathy, myocardial injury/myocarditis, cardiac arrhythmias, cardiogenic shock, myocardial ischemia and acute pulmonale; vascular complications including deep vein thrombosis, pulmonary embolism and catheter-related thrombosis; endocrine disorders including hyperglycemia and diabetic ketoacidosis; acute renal injuries; hepatic dysfunctions, gastrointestinal abnormalities and neurologic issues such as encephalopathy, stroke and Guillain-Barrie syndrome. The effects of COVID-19 on multiple organs could potentially produce distinct volatile metabolites and their signature patterns between COVID-19 negative and positive patients, and between symptomatic and asymptomatic COVID-19 patient groups. Hence, noninvasive exhaled breath analysis could provide an urgently needed screening method to detect pre-symptomatic and asymptomatic COVID-19 patients, monitor the progression and assess therapeutic efficacy in this pandemic and future outbreak.

Breath analysis is a developing modality with great potential to simplify the workup of suspected diseases including cancers and bacterium and virus infections. In the past fifteen years, the analysis of human breath has become an international research frontier because of its potential applications in disease diagnosis and metabolomics. Several methods have been developed for analysis of exhaled breath including “electronic nose” or gas sensor array, proton-transfer reaction mass spectrometry (PTR-MS), selected ion flow tube mass spectrometry (SIFT-MS), and gas chromatography-mass spectrometry (GC-MS). An electronic nose is an array of chemiresistive sensors that cannot be used to identify specific compounds. The recently developed thiol-modified gold nanoparticle sensor arrays for breath analysis to detect COVID-19 patients, published by Haick et al. in Israel cannot recognize specific VOCs and suffers interferences by the inorganic gases and VOC mixtures in exhaled breath, which results in low diagnostic accuracy and specificity. Field asymmetric ion mobility spectrometry (FAIMS) for breath analysis, developed by Owlstone Medical Inc. in England, is similar to the electronic nose for providing electrical signals and cannot be used to perform measurements of specific VOCs. Both PTR-MS and SIFT-MS measurements do not provide as much information as GC-MS. Among the developed analytical methods, GC-MS is the most widely used technique for analysis of VOCs. Since VOCs in breath are excreted at parts per billion per volume (ppbv) to parts per trillion per volume (pptv) concentrations, a preconcentration process is typically required to increase VOC concentrations to above the detection limits for breath analysis by GC-MS. Conventional preconcentrators used for GC-MS consist of stainless steel or glass-capillary tubes packed with one or more granular absorbent materials. The preconcentration process requires further cryogenic concentration to attain the necessary focus (injection volumes and times) for GC-MS analysis. Preconcentration based on physical adsorption using solid phase microextraction (SPME) fibers and sorbent-in-needle traps is a widely used method for breath analyses. However, SPME and sorbent-in-needle traps are limited to preconcentrating VOCs that can adsorb on the adsorbents. More recent preconcentrators fabricated on silicon wafers using microelectromechanical system (MEMS) technology consist of a granular adsorbent packed in a microfluidic channel. All preconcentration methods have physical adsorption/thermal desorption hysteresis constraints. The analysis of exhaled breath condensate (EBC) method requires low temperatures (below −25° C.) to condense the breath matrix. EBC method has disadvantages of unknown breath sample volume and inability to condense very volatile small compounds in breath.

U.S. Pat. Nos. 11,016,082 and 9,638,695 disclose certain methods for detecting cancer disease states by forming adducts between a reactive compound and carbonyl-containing VOCs in exhaled breath samples, quantifying adducts to establish subject values, and comparing the subject values to corresponding healthy specimen threshold values, wherein the presence of subject values greater than their respective healthy specimen threshold values indicate a cancer disease state. While effective, these methods are reliant upon relatively large, stationary and expensive equipment such as high performance liquid chromatography coupled with mass spectrometry (HPLC-MS), gas chromatography coupled with mass spectrometry (GC-MS), or Fourier transform ion cyclotron resonance-mass spectrometry (FT-ICR-MS) for quantification and analysis. A need exists for new devices and methods for breath collection and analysis with portable equipment to facilitate point-of-care diagnoses, and in particular, inexpensive devices and methods for monitoring changes in the carbonyl metabolome to create a mobile breath analysis technology for rapid screening of COVID-19 patients and other purposes.

### SUMMARY

The invention addresses this unmet need by providing a novel mechanism for capturing target compounds in a gaseous sample, such as VOCs in an exhaled breath sample. This breath collection tool may facilitate detection of an infection by SARS-CoV-2 by adapting a proven breath analysis technology that profiles trace volatile carbonyls exhaled at ppbv to pptv levels. Changes in the carbonyl-metabolome correlate closely with shifts in cellular biochemical processes. The role of viral-induced oxidative stress leading to volatile carbonyl formation has been clearly established, particularly in acute respiratory viral infections. Viral infections produce both common volatile organic compounds (VOCs) and unique VOC fingerprints corresponding to three subtypes of influenza viruses, respiratory syncytial virus, and rhinovirus infected cells. Thus, an approach that chemoselectively isolates carbonyl compounds from exhaled breath at concentrations as low as the picomolar range is ideally suited to monitor even subtle changes in carbonyl production, such as might be expected with SARS-CoV-2 patients that are pre-symptomatic and asymptomatic.

Portable GC analysis of breath samples collected using silicon microreactor technology may be used to predict SARS-CoV-2 infection. There are approximately one hundred saturated and unsaturated aldehydes and ketones in human exhaled breath, including saturated aldehydes, ketones, hydroxy-aldehydes, hydroxy-ketones, and unsaturated 2-alkenals, 4-hydroxy-2-alkenals, and 4-hydroxy-2,6-alkadienals. Some aldehydes are metabolites related to COVID virus infection for symptomatic and asymptomatic patients. The combination of a microchip-based breath analyzer technology with a portable GC and photoionization detector (PID) for analysis could be transformative for screening of COVID-19 patients for several reasons: the clinical and social importance of a rapid and accurate screening of COVID-19 patients is immediate; the method is simple and straightforward; and most importantly, the detection sensitivity and specificity of COVID-19 could be comparable with current standard tests. Breath analysis is noninvasive and takes a few minutes using state-of-the-art microchip technology.

In one embodiment, the present invention is an apparatus for capturing one or more target compounds in a gaseous sample, the apparatus comprising a microreactor including a first side and a second side opposite the first side, a circular recess formed in the first side, the circular recess including a central area and a perimeter, a plurality of vent holes located within the recess, the vent holes spaced along the perimeter and extending through the microreactor, and a plurality of micropillars positioned within the recess between the central area and the perimeter, the plurality of micropillars forming a plurality of microfluidic channels between the central area and the perimeter; wherein the plurality of micropillars are coated with a capture material capable of capturing the one or more target compounds. In further embodiments, the apparatus includes an apparatus body including a hollow interior, an inlet and an outlet; wherein the microreactor is located within the interior in fluid communication with the inlet and outlet, such that airflow into the inlet is directed toward the central area of the first side, is redirected radially through the plurality of microfluidic channels, through the plurality of vent holes, and exits the body through the outlet. In certain embodiments, the apparatus body includes an upper section and a lower section, the upper section and lower section being removably attachable to each other; wherein the upper section includes an upper body having an upper end including the inlet, a lower end, at least one side between the upper end and the lower end, and a first passageway extending from the inlet, through the upper body, and terminating in a nozzle opposite the inlet; wherein the lower section includes a lower body having an upper end, a lower end, at least one side between the upper end and the lower end, and a tube extending from the lower end and terminating in the outlet, and a second passageway extending from the upper end, through the lower body, and terminating in the outlet; wherein the nozzle is inserted at least partially within the upper end of the lower body; and wherein the first passageway and second passageway cooperatively form the hollow interior of the apparatus body. In some embodiments, the apparatus includes a tube extending from the upper end of the upper body, wherein the tube terminates in the inlet. In further embodiments, the microreactor is positioned within a cavity in the second passageway sized and shaped to receive the microreactor between the nozzle and the outlet. In further embodiments, the apparatus includes a cover attached to the first side of the microreactor, the cover including an entry port aligned over the central area, wherein the circular recess and cover cooperatively define an interior of the microreactor. In some embodiments, airflow into the inlet sequentially passes through the first passageway, nozzle, entry port, interior of the microreactor, vent holes of the microreactor, second passageway, and outlet. In certain embodiments, the nozzle engages and is received by the entry port of the cover. In further embodiments, the plurality of micropillars includes micropillars of different diameters, wherein micropillars with larger diameters are located closer to the central area than micropillars with smaller diameters, and wherein micropillars with smaller diameters are located closer to the perimeter than micropillars with larger diameters. In certain embodiments, spacing between micropillars in the plurality of micropillars varies, with greater spacing between micropillars in proximity to the central area than between micropillars in proximity to the perimeter. In some embodiments, micropillars in the plurality of micropillars are arranged such that radial airflow from the central area to any of the plurality of vent holes is deflected around multiple micropillars. In further embodiments, the apparatus includes an apparatus body including a hollow interior, an inlet and an outlet; wherein the microreactor is located within the interior in fluid communication with the inlet and outlet, such that airflow into the inlet is directed toward the central area of the first side, is redirected radially through the plurality of microfluidic channels, through the plurality of vent holes, and exits the body through the outlet.

In another embodiment, the present invention is an apparatus for capturing one or more target compounds in a gaseous sample, the apparatus comprising an apparatus body including an upper section and a lower section, the upper section and lower section being removably attachable to each other; wherein the upper section includes an upper body having an upper end including an inlet, a lower end, at least one side between the upper end and the lower end, and a first passageway extending from the inlet, through the upper body, and terminating in a nozzle opposite the inlet; wherein the lower section includes a lower body having an upper end, a lower end, at least one side between the upper end and the lower end, and a tube extending from the lower end and terminating in an outlet, and a second passageway extending from the upper end, through the lower body, and terminating in the outlet; wherein the nozzle is inserted at least partially within the upper end of the lower body; and wherein the first passageway and second passageway cooperatively form a hollow interior of the apparatus body; and a microreactor within the second passageway in fluid communication with the inlet and outlet, such that airflow into the inlet sequentially passes through the first passageway, nozzle, microreactor, second passageway, and outlet. In some embodiments, the microreactor is accessible when the lower section is separated from the upper section. In further embodiments, the apparatus includes a tube extending from the upper end of the upper body, wherein the tube terminates in the inlet. In certain embodiments, the microreactor is positioned within a cavity in the second passageway sized and shaped to receive the microreactor between the nozzle and the outlet. In some embodiments, microreactor includes a first side and a second side opposite the first side, a circular recess formed in the first side, the circular recess including a central area and a perimeter, a plurality of vent holes located within the recess, the vent holes spaced along the perimeter and extending through the microreactor, and a plurality of micropillars positioned within the recess between the central area and the perimeter, the plurality of micropillars forming a plurality of microfluidic channels between the central area and the perimeter; wherein the plurality of micropillars are coated with a capture material capable of capturing the one or more target compounds. In further embodiments, the apparatus includes a cover attached to the first side of the microreactor, the cover including an entry port aligned over the central area, wherein the circular recess and cover cooperatively define an interior of the microreactor. In certain embodiments, airflow into the inlet sequentially passes through the first passageway, nozzle, entry port, interior of the microreactor, second passageway, and outlet. In some embodiments, the nozzle engages and is received by the entry port of the cover.

In yet another embodiment, the present invention is a method of capturing one or more target compounds in a gaseous sample, the method comprising providing an apparatus as described herein; directing an airflow of the gaseous sample toward the central area, such that the airflow contacts the central area and is redirected through the plurality of microfluidic channels formed by the plurality of micropillars and into the vent holes; and capturing the one or more target compounds using the capture material on the plurality of micropillars. In some embodiments, the method further includes eluting the one or more target compounds from the plurality of micropillars using an eluant. In further embodiments, the eluant is methanol. In certain embodiments, the method includes, after said eluting, determining a binary outcome based on the captured one or more target compounds. In some embodiments, the gaseous sample is an exhaled breath sample, wherein the one or more target compounds are carbonyl-containing volatile organic compounds, and wherein the binary outcome is the presence or absence of a disease state in the provider of the exhaled breath sample. In further embodiments, the apparatus further comprises a cover attached to the first side of the microreactor, the cover including an entry port aligned over the central area, and wherein the circular recess and cover cooperatively define an interior of the microreactor; and wherein said eluting includes introducing the eluant into the interior of the microreactor through the entry port. In certain embodiments, the capture material is a reactive compound, and wherein the capturing comprises forming adducts between the reactive compound and the one or more target compounds. In some embodiments, the capture material is an adsorptive material. In further embodiments, the one or more target compounds are carbonyl-containing volatile organic compounds. In certain embodiments, the gaseous sample is an exhaled breath sample. In some embodiments, the method includes determining a binary outcome based on the captured one or more target compounds. In further embodiments, the gaseous sample is an exhaled breath sample and wherein the binary outcome is the presence or absence of a disease state in the provider of the exhaled breath sample. In certain embodiments, the disease state is COVID-19. In some embodiments, the determining is performed using a machine learning system based on concentrations or ratios of concentrations of the captured one or more target compounds as input parameters to the machined learning system.

In yet another embodiment, the present invention is a method of capturing one or more target compounds in a gaseous sample, the method comprising providing an apparatus as described herein; directing an airflow of the gaseous sample into the inlet and sequentially through the first passageway, nozzle, microreactor, second passageway, and outlet; and capturing the one or more target compounds using a capture material within the microreactor. In some embodiments, the method includes eluting the one or more target compounds from the microreactor using an eluant. In further embodiments, the eluant is methanol. In certain embodiments, the eluant is introduced into the apparatus through the inlet and exits the apparatus through the outlet. In some embodiments, the eluant is introduced into the microreactor without removing the microreactor from the apparatus. In further embodiments, the eluant is introduced into the microreactor after removing the microreactor from the apparatus. In certain embodiments, the capture material is a reactive compound, and wherein the capturing comprises forming adducts between the reactive compound and the one or more target compounds. In some embodiments, the capture material is an adsorptive material. In further embodiments, the one or more target compounds are carbonyl-containing volatile organic compounds. In certain embodiments, the gaseous sample is an exhaled breath sample. In some embodiments, the method includes determining a binary outcome based on the captured one or more target compounds. In further embodiments, the gaseous sample is an exhaled breath sample and wherein the binary outcome is the presence or absence of a disease state in the provider of the exhaled breath sample. In certain embodiments, wherein the disease state is COVID-19. In some embodiments, the determining is performed using a machine learning system based on concentrations or ratios of concentrations of the captured one or more target compounds as input parameters to the machined learning system.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1A depicts a first embodiment of a breath collection tool.

FIG. 1B depicts the first embodiment of the breath collection tool separated into an upper section and a lower section.

FIG. 1C depicts a cross-sectional view of the first embodiment of the breath collection tool along line C-C, with the upper section and lower section spaced apart.

FIG. 2A depicts a side view of a second embodiment of a breath collection tool.

FIG. 2B depicts the breath collection tool of FIG. 2A separated into an upper section and a lower section.

FIG. 2C depicts a lower end perspective view of the breath collection tool of FIG. 2B.

FIG. 2D depicts an upper end perspective view of the breath collection tool of FIG. 2B.

FIG. 2E depicts a partial cross-sectional view of the breath collection tool of FIG. 2A.

FIG. 2F depicts an expanded view of the interface between the nozzle, gasket, cover and microreactor in FIG. 2E.

FIG. 3A depicts an upper end perspective view of a third embodiment of a breath collection tool with optional mouthpiece attached.

FIG. 3B depicts a partial cut-away view of the breath collection tool of FIG. 3A.

FIG. 3C depicts a side cut-away view of the breath collection tool of FIG. 3A, omitting the optional mouthpiece.

FIG. 4A depicts a top perspective view of the microreactor and cover.

FIG. 4B depicts the microreactor of FIG. 4A, omitting the cover and including arrows illustrating airflow through the microreactor.

FIG. 5 is schematic illustration of a micropillar coated with an exemplary reactive compound, ATM, and the chemical reaction between the ATM coating and a carbonyl-containing VOC to capture the carbonyl-containing VOC as a non-volatile adduct.

FIG. 6A is a schematic illustration of a micropillar coated with reactive compound, AMAH, and the chemical reaction between the AMAH coating and a carbonyl-containing ketone or aldehyde to capture the chemical as non-volatile adduct.

FIG. 6B is a schematic illustration of the chemical reaction between a charged AMAH-carbonyl adduct and PVP to form a neutral AMAH-carbonyl adduct.

FIG. 6C summarizes the process of reacting an AMAH coating with a carbonyl-containing VOC in a breath sample within the microreactor, subsequent sample processing by eluting the adduct from the microreactor, and reacting the AMAH-carbonyl adduct with a basic polymer to form a volatile neutral AMAH-carbonyl adduct, and injecting the volatile adduct into a GC-MS or GC-PID for analysis.

FIG. 7 displays a panel of exemplary aminooxy and hydrazine reactive compounds capable of forming adducts with carbonyl-containing VOCs.

FIG. 8 depicts a reactive compound, PFBHA⋅HCL, and the chemical reaction between PFBHA and a carbonyl-containing VOC to capture the carbonyl-containing VOC as a non-volatile adduct.

FIG. 9 is a series of GC-MS chromatographs of COVID-positive (top two chromatographs) and COVID-negative (bottom four chromatographs) subjects.

FIG. 10 is series of box plots comparing levels of specific carbonyl-containing VOCs (acetone, top left; 2-butanone, top right; pentanal, bottom left; nonanal/(C4-C9) bottom right) in COVID-positive and COVID-negative subjects.

FIG. 11 is a graph comparing the capture efficiency of the breath collection tool over three repeated runs of gaseous samples including propanal d2, butanone d5, pentanone d5 or hexanal d12 at a flow rate of 375 mL/min.

FIG. 12 is a series of GC-MS chromatographs indicating the effects of repeated exhaled breaths into the breath collection tool. Peaks indicating the presence of acetone and acetone-d6 (i.e., acetone with six hydrogen atoms replaced with deuterium atoms as internal reference) are labeled.

FIG. 13 is a series of GC-MS chromatographs showing collected carbonyls from exhaled breath samples from COVID-positive (top two chromatographs) and COVID-negative (bottom two chromatographs) subjects

FIG. 14 is series of box plots comparing levels of specific carbonyl-containing VOCs (hexanal, top left; nonanal, top middle; nonanone/OT, top right; 2-butanone/OT, bottom left; octanone/OT, bottom middle; dodecanone/OT bottom right) in COVID-positive and -negative subjects.

FIG. 15 is series of box plots comparing levels of specific carbonyl-containing VOCs or combinations (C1+C2+C3, top left; decanal, top middle; acetone, top right; nonanone/OT, bottom left; nonanone, bottom middle; decanal/OT bottom right) in COVID-positive and COVID-negative subjects.

FIG. 16 includes a pair of graphs depicting the results of machine learning (ML) algorithms for detection of positives of 55 alpha, 86 delta and 181 negatives by UHPLC-MS analysis of exhaled breath samples. The left graph depicts the ROC curve for analysis of exhaled breath samples by measurement of acetone/butanone and pentanal/OT. The area under the curve for the left graph is 0.9647. The right graph depicts the ROC curve for analysis of exhaled breath samples by measurement of acetone, butanal/OT, (C1+C2+C3), hexanal, and acetone/butanone. The area under the curve for the right graph is 0.9327.

FIG. 17 displays overlaid chromatographs from a portable GC-PID showing exhaled breath samples from two COVID-19 positive subjects (solid lines) and two COVID-19 negative subjects (dashed lines).

FIG. 18 is series of box plots comparing levels of specific carbonyl-containing VOCs (acetone, top left; propanal, top middle; pentanal, top right; 2-butanone, bottom left; 2-pentanone/OT, bottom middle; and pentanal/2-pentanone, bottom right) in COVID-positive and COVID-negative subjects.

### DETAILED DESCRIPTION OF THE PREFERRED EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter. As used herein, the term “about,” when referring to a value or to an amount is meant to encompass variations of ±10% of the most precise digit in the value or amount (e.g., “about 1” refers to 0.9 to 1.1, “about 1.1” refers to 1.09 to 1.11, etc.).

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter relates to a novel disposable breath collection tool and use thereof with a portable GC with photoionization detector (PID) for mobile analysis of a group of carbonyl compounds in exhaled breath. This breath collection tool may be used for rapid screening of COVID-19 patients or patients with other disease states as known in the art to affect the composition of carbonyl compounds in exhaled breath. While the effectiveness of the breath collection tool is shown herein primarily in terms of collecting and concentrating carbonyl-containing VOCs from exhaled breath samples for COVID-19 screening, it should be understood that the tool may be used to detect a variety of disease states with varying analysis of detected carbonyl-containing VOCs. The breath collection tool may also be used to collect and concentrate target compounds other than carbonyl-containing VOCs in exhaled breath by varying the capture material within the microreactor. For example, the tool may also be used to determine whether an individual has consumed alcohol above the legal limit for operating a motor vehicle by using a capture material competent to capture alcohols in the individual's exhaled breath. For another example, the tool may be used to determine whether an individual has consumed Cannabis products by using a capture material competent to capture cannabinoids in the individual's exhaled breath. The breath collection tool may further be used to collect target compounds in gaseous samples apart from exhaled breath. For example, the tool could be used in air quality testing by directing an airflow of ambient air into the tool and including a capture material within the microreactor that competently and reversibly captures an air contaminant of interest. Put another way, the breath collection tool may be used to capture and facilitate determining a binary outcome (e.g., presence or absence of a disease state, such as COVID-19, presence or absence of alcohol or Cannabis in excess of a legal limit, presence or absence of an air contaminant above a predetermined level, etc.) based on analysis of one or more target compounds captured by the tool.

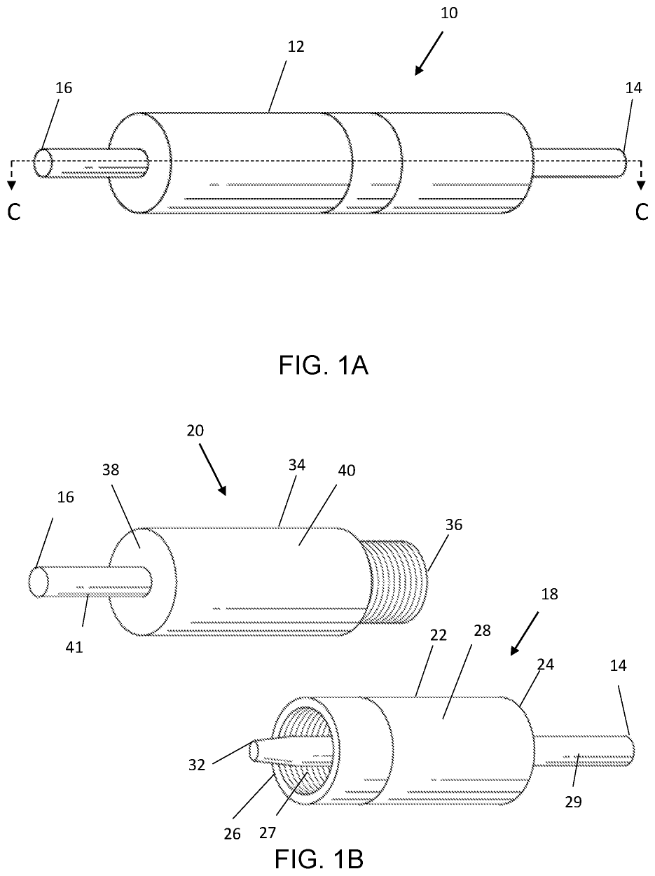

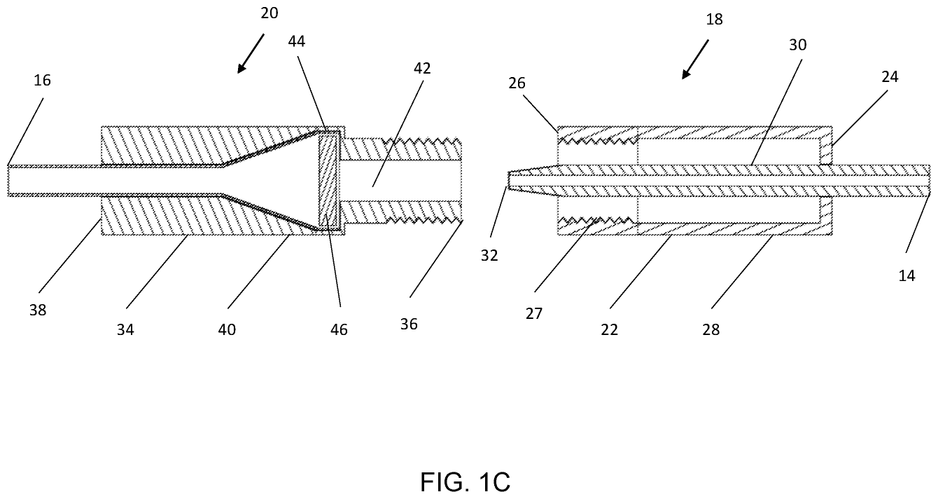

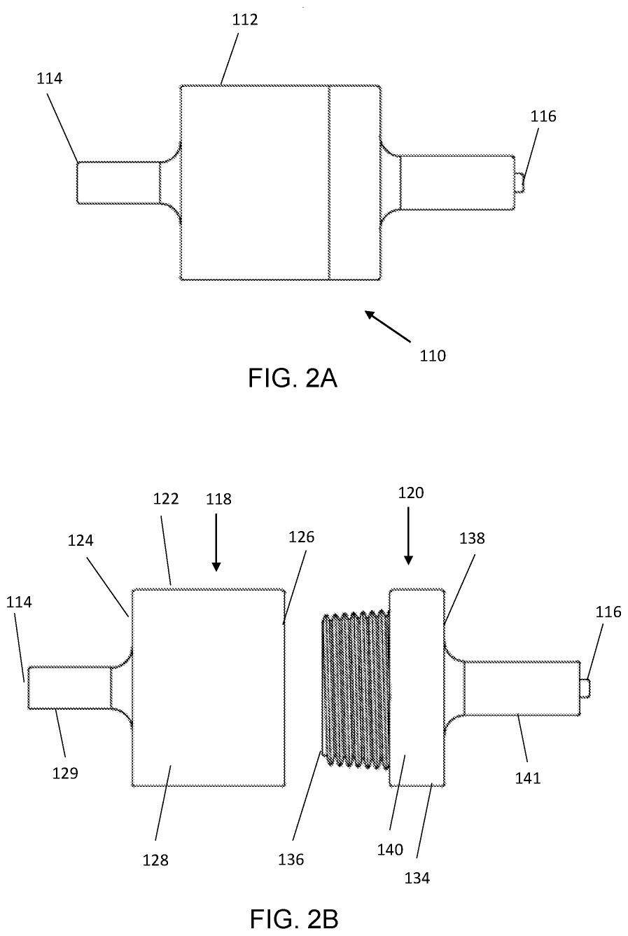

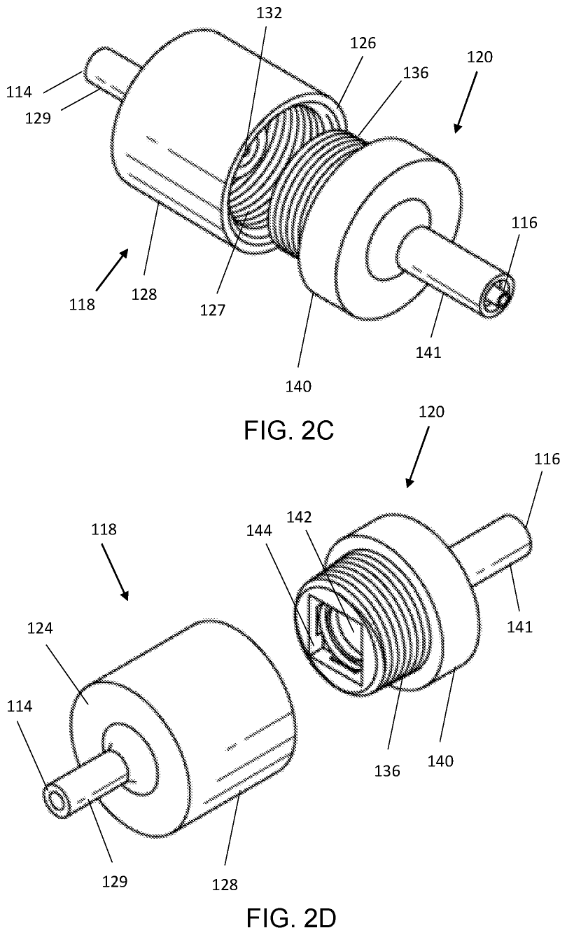

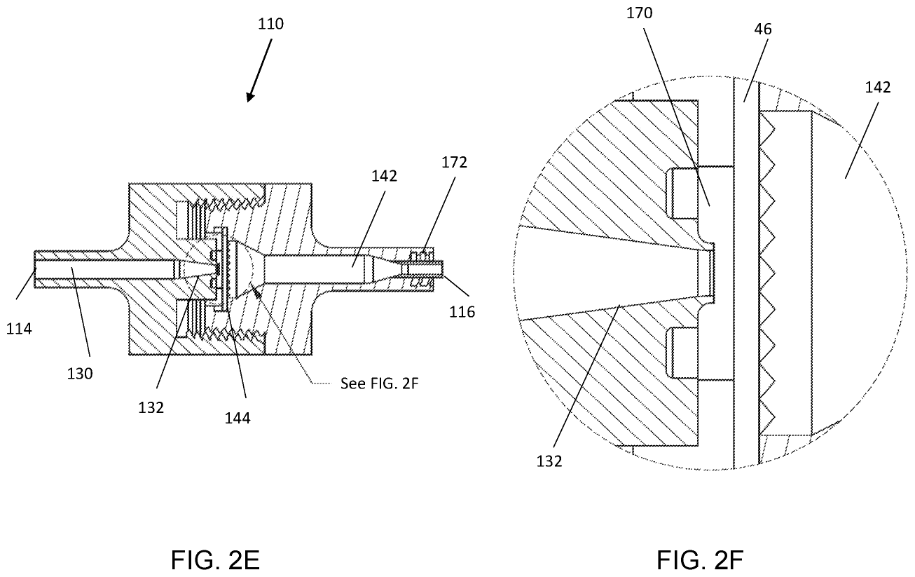

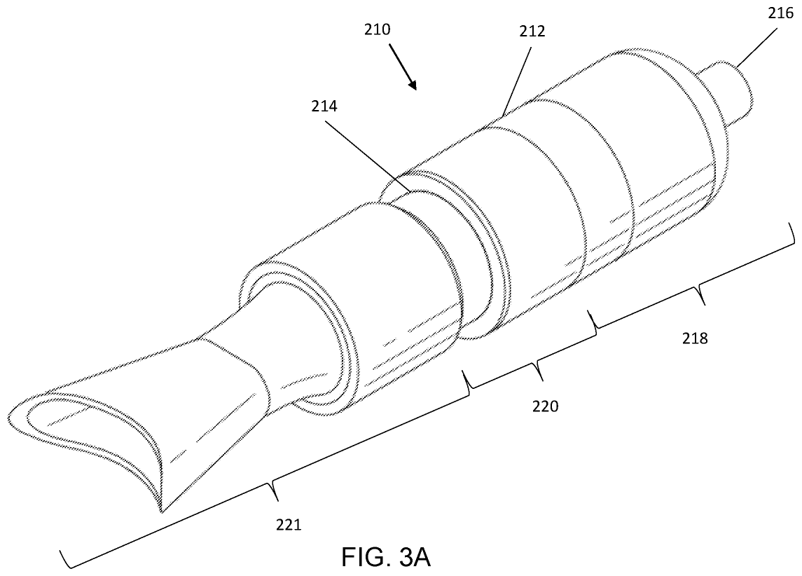

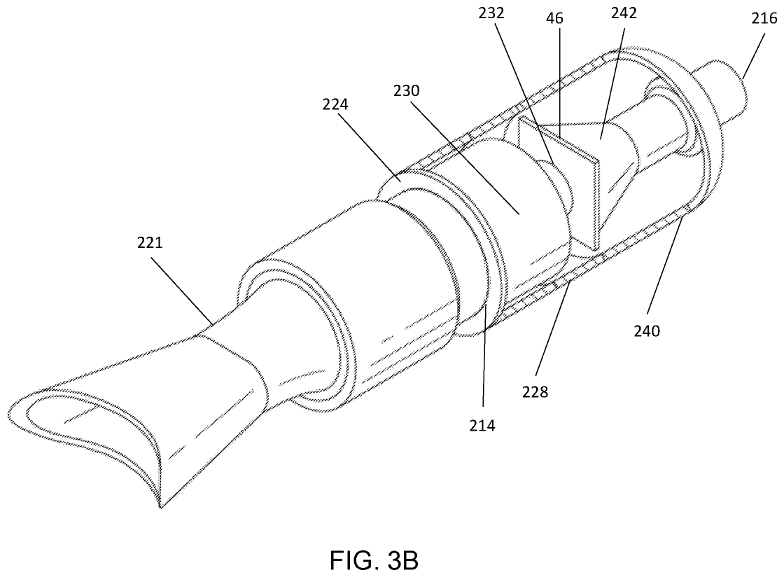

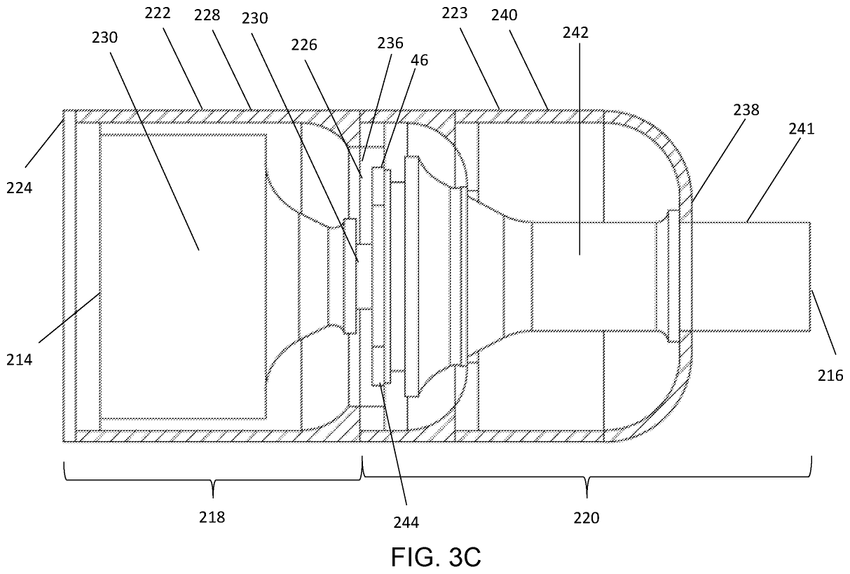

A first embodiment of a breath collection tool 10, as shown in FIGS. 1A-1C, and a second embodiment 110 as shown in FIGS. 2A-2E, and a third embodiment 210 as shown in FIGS. 3A-3C each include an apparatus body 12, 112, 212 including an inlet 14, 114, 214 and an outlet 16, 116, 216 wherein the inlet 14, 114, 214 and outlet 16, 116, 216 are in fluid communication through the apparatus body 12, 112, 212. The apparatus body 12, 112, 212 is separable into an upper section 18, 118, 218 and a lower section 20, 120, 220 which are removably coupled via a threaded connection, luer lock, slip tip, snap fit connection, or other attachment means as known in the art. In some embodiments, an optional mouthpiece 221 is removably attached to the inlet 214 to facilitate exhaling into the breath collection tool 210.

The upper section 18, 118, 218 includes a generally cylindrical upper body 22, 122, 222 including an upper end 24, 124, 224 an open lower end 26, 126, 226 and at least one side 28, 128, 228 extending between the upper end 24, 124, 224 and open lower end 26, 126, 226. The open lower ends 26, 126 of the first and second embodiments 10, 110 each include a threaded interior 27, 127 for engaging the lower section 20, 120. In contrast, the open lower end 226 of the third embodiment 210 lacks a threaded interior and engages the lower section 220 via a snap fit connection. The first and second embodiments 10, 110 each include a tube 29, 129 extending from the upper end 24, 124 and terminating in the inlet 14, 114. In contrast, the inlet 214 of the third embodiment 210 is located in the upper end 224. As most easily seen in FIGS. 1C, 2E and 3C, the upper section 18, 118, 218 includes a generally cylindrical first passageway 30, 130, 230 extending from the inlet 14, 114, 214 and terminates in a nozzle 32, 132, 232 opposite the inlet 14, 114, 214.

The lower section 20, 120, 220 includes a generally cylindrical lower body 34, 134, 234 having an upper end 36, 136, 236, a lower end 38, 138, 238, at least one side 40, 140, 240 extending between the upper end 36, 136, 236 and lower end 38, 138, 238 and a tube 41, 141, 241 extending from the lower end 38, 138, 238 and terminating in the outlet 16, 116, 216. The upper ends 36, 136 of the first and second embodiments 10, 110 are threaded for engaging the respective threaded interiors 27, 127 of the upper sections 18, 118. In contrast, the upper end 236 of the third embodiment 210 is not threaded and engages the upper section 218 via a snap fit connection. A generally cylindrical second passageway 42, 142, 242 extends from the open upper end 36, 136, 236 through the lower body 34, 134, and tube 41, 141, 241 to terminate in the outlet 16, 116, 216. The second passageway includes a cavity 44, 144, 244 sized and shaped to receive a microreactor 46, as described below, and narrows between the cavity 44, 144, 244 and the outlet 16, 116, 226. As most easily seen in FIGS. 2D and 3C, the cavity 144, 244 is readily accessible when the upper section 118, 218 and lower section 120, 220 are separated. In the first embodiment, the lower section 20 may be formed of multiple separable parts which, when separated, allow access to the cavity 44 therein.

In the second embodiment of the breath collection tool 110, as most easily seen in FIG. 2E, a threaded recess 172 radially surrounds the second passageway 142 at the outlet 116. The threaded recess 172 allows for threaded coupling between the breath collection tool 110 and a downstream element, such as a filter unit or an inlet line for a pump or a gas chromatograph.

When the upper section 18, 118, 218 and lower section 20, 120, 220 are attached to each other, the nozzle 32, 132, 232 extends into the second passageway 42, 142, 242 in the direction of the cavity 44, 144, 244. In the first and second embodiments, 10, 110, when the upper section 18, 118 and lower section 20, 120 are attached, the threaded interior 27, 127 of the upper section 18, 118 receives and engages the threaded upper end 36, 136 of the lower section 20, 120. When the upper section 18, 118, 218 and lower section 20, 120, 220 are attached, the first passageway 30, 130, 230 and second passageway 42, 142, 242 cooperatively form a hollow interior to the body 12, 112, 212 such that airflow into the inlet 14, 114, 214 passes sequentially through the first passageway 30, 130, 230, the nozzle 32, 132, 232, the second passageway 42, 142, 242 including the cavity 44, 144, 244, and exits via the outlet 16, 116, 216.

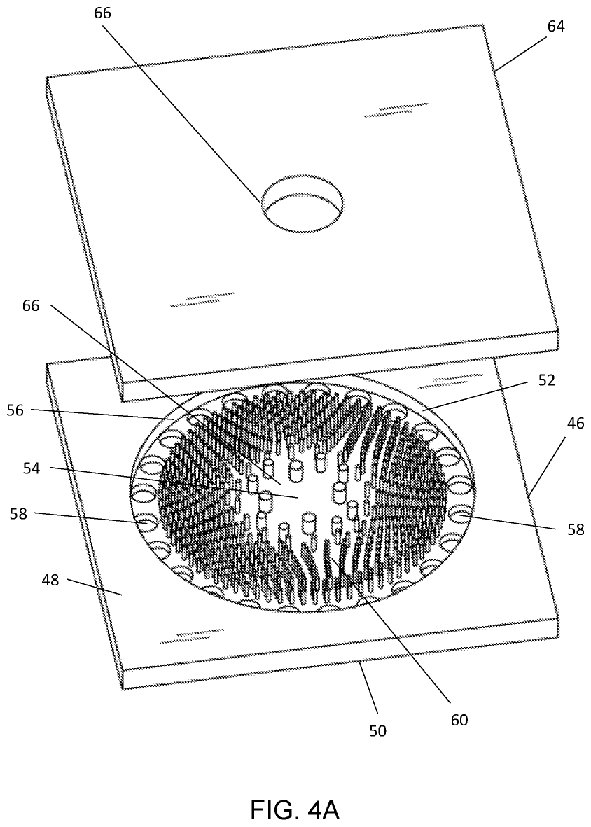

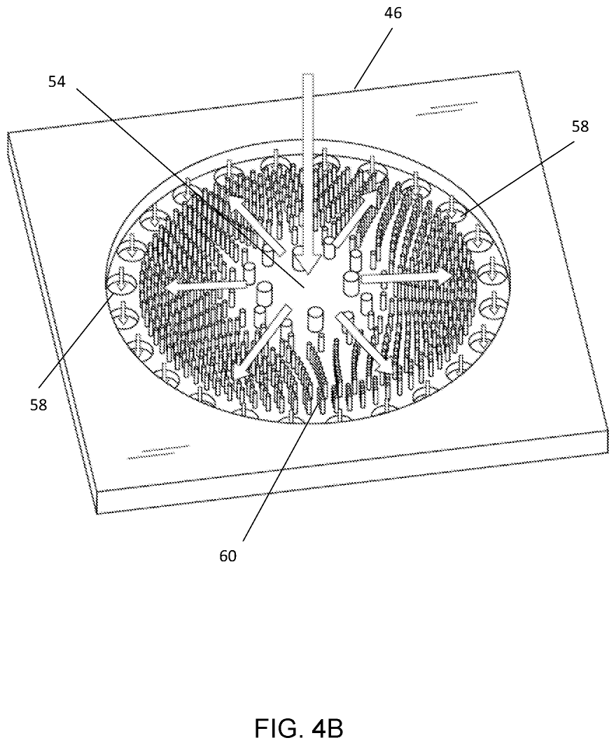

Referring now to FIGS. 4A and 4B, the first and second embodiments of a breath collection tool 10, 110, 210 each include a silicon microreactor 46 of appropriate dimensions, such as, for example, 18 mm×18 mm×1 mm or 18 mm×18 mm×0.5 mm, for insertion into the cavity 44, 144, 244 in the lower section 20, 120, 220 the cavity 44, 144, 244 being shaped to receive the microreactor 46. After the microreactor 46 is installed, the upper section 18, 118, 218 is attached to the lower section 20, 120, 220 to ready the breath analyzer tool 10, 110, 210 for use.

The microreactor 46 includes a first side 48 and a second side 50 opposite the first side 48, and a circular recess 52 formed in the first side, the circular recess 52 including a central area 54 and a perimeter 56. A plurality of vent holes 58 are located within the recess 52, spaced along the perimeter 56 and extending through the microreactor 46. A plurality of spaced apart micropillars 60 are positioned within the recess between the central area 54 and the perimeter 56, forming a plurality of microfluidic channels 62 between the central area 54 and the perimeter 56. Computational fluid dynamics (CFD) was used to simulate the micropillar patterns for uniform laminar flow air along radial direction. In some embodiments, the micropillars 60 are arranged in a pattern, such as a unilateral triangular pattern, such that radial airflow from the central area 54 cannot proceed in a straight line toward the perimeter 56 but is deflected around multiple micropillars 60.

In some embodiments, the microreactor 46 is fabricated on silicon wafers using MEMS technologies. The microfluidic channels 62 and the micropillars 60 are created by deep reactive ion etching (DRIE) and are sealed by bonding a cover 64 on the first side 50 of the microreactor 46. In some embodiments, the cover 64 is a glass wafer or an elastic polydimethyl-silicone (PDMS) polymer, and includes a central entry port 66 with a diameter of, in some embodiments, about 2 mm. The circular recess 52 and cover 64 define an interior 68 of the microreactor 46, wherein the entry port 66 and vent holes 58 provide access to the interior 68 of the microreactor 46. The entry port 66 is aligned over the central area 54 of the microreactor 46. When the microreactor 46 is installed in the breath collection tool 10, 110, 210 and an airflow (such as an exhaled breath) enters the inlet 14, 114, 214, the airflow passes sequentially through the first passageway 30, 130, 230 the nozzle 32, 132, 232 and the entry port 66 of the cover 64 to enter the interior 68 of the microreactor 46. As shown in FIG. 4B, the entering airflow then contacts the central area 54 and is redirected radially outwards, passing through the microfluidic channels 62 formed between the micropillars 60, and exiting the interior 68 through the plurality of vent holes 58. The airflow than continues onwards through second passageway 42, 142, 242 and exits the breath collection tool 10, 110, 210 through the outlet 16, 116, 216. In some embodiments, as most easily seen in FIG. 2E, the nozzle 132 engages and is received by the entry port 66. In certain embodiments, a gasket 170 radially surrounds the interface between the nozzle 132 and the entry port 66 to minimize leakage between the nozzle 132 and the microreactor 46.

The size and spacing of the micropillars 60 is based on competing factors. Providing a plurality of micropillars 60 with comparatively smaller diameter and spacing the micropillars 60 closely together increases the reactive surface of the microreactor 46, facilitating capture and concentration of target compounds in a gaseous sample, such as carbonyl-containing VOCs in an exhaled breath sample. However, when micropillars 60 are too densely packed, users had difficulty exhaling their breath with sufficient force to flow through the entirety of the breath collection tool. To address this issue, in certain embodiments, the micropillars 60 are arranged on a gradient. Micropillars 60 located in proximity to the central area 54 have comparatively larger diameters and are spaced comparatively far apart from each other. Micropillars 60 located in proximity to the perimeter 56 have comparatively smaller diameters and less spacing between each micropillar, such that the smallest diameter and most densely packed micropillars 60 are closest to the perimeter 56 and the largest diameter and most spaced micropillars 60 are closest to the central area 54.

While the size of micropillars 60 is discussed in terms of their diameters, it should be understood that micropillars are not necessarily cylindrical in shape with circular cross-sections, and may have square, diamond, oval, or other geometrically-shaped cross-sections, or may be fin-shaped. In some embodiments, the spacing between the most densely packed micropillars is within the range of 30 μm to 150 μm and the spacing between the least densely packed micropillars is within the range of 200 μm to 300 μm. In further embodiments, the diameter of each micropillar is within the range of 50 μm to 200 μm. In certain embodiments, the height of each micropillar is within the range of 200 μm to 600 μm. In some embodiments, each micropillar has a substantially uniform height and the depth of the circular recess is equal to the height of the micropillars. These parameters may be varied for different applications, as the spacing, diameter and height of micropillars affects the resistance for flowing air through the microreactor and the capture efficiencies of target compounds.

The plurality of micropillars 60 are coated with a capture material capable of retaining one or more target compounds within the microreactor. In some embodiments, the capture material is an adsorptive material, such as a porous polymer film, capable of adsorption of one or more target compounds in a gaseous or liquid sample passed through the microreactor. In other embodiments, as discussed in further detail, the capture material is a reactive compound capable of forming adducts with one or more target compounds in a gaseous or liquid sample passed through the microreactor. In some embodiments, the target compounds are carbonyl-containing VOCs. In some embodiments, the reactive compound is capable of forming adducts with carbonyl-containing VOCs. In certain embodiments, the surface of the micropillars is a metal oxide, such as silicon dioxide, aluminum dioxide, titanium dioxide, or others. The metal oxide surface provides a support to attach the capture material which, in some embodiments, is a reactive compound, such as an aminooxy compound or hydrazine compound.

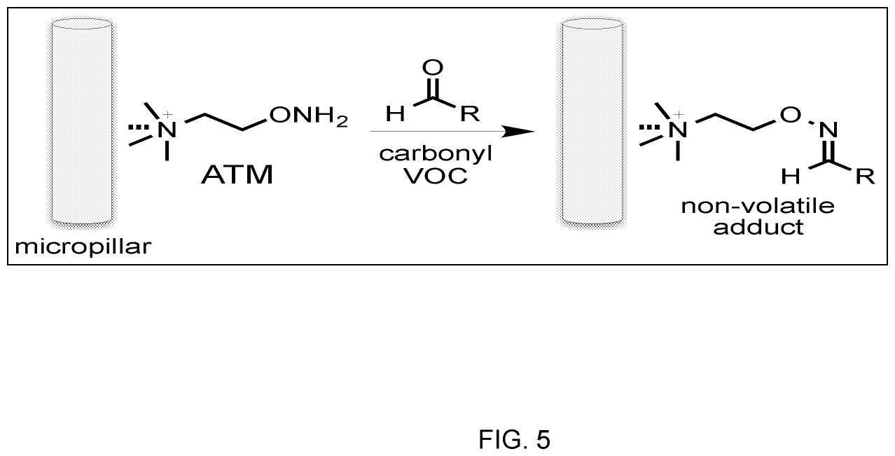

FIG. 5 shows a schematic illustration of a micropillar coated with an exemplary capture material, 2-(aminooxy)ethyl-N,N,N-trimethylammonium (ATM), and the chemical reaction between the ATM coating and a carbonyl-containing VOC to capture the carbonyl-containing VOC as a non-volatile adduct. In some embodiments, the cationic reactive compound may be paired with an anion, such as, for example, triflate. The micropillars 60 provide surface area for the coating and distribute air flowing through the microreactor 46. In some embodiments, the reactive compound coating is applied to the surfaces of the micropillars 60 by flowing the reactive compound in methanol into the microreactor 46 and then evaporating methanol in a vacuum oven. In some embodiments, the microfluidic volume of the interior 69 of the microreactor 46 is about 20 μL. Conventional silica bead packed tubes have internal volumes of at least a few milliliters, so the smaller interior volume of the microreactor 46 is more effective for preconcentration of analytes from a liter-sized breath sample to a microliter-sized liquid solution.

In use, the assembled breath collection tool 10, 110, 220 may optionally be fitted with a disposable mouthpiece 221 on the inlet 14, 114, 214 and a filter unit (not shown) on the outlet 16, 116, 216 via luer lock type fittings or other attachment means as known in the art. In some embodiments, the filter is rated HEPA H13 or higher to trap viruses, serving as a safety feature to protect health care workers administering the breath test. The breath collection then can proceed by having the subject exhale into the mouthpiece in a continuous breath. The exhaled breath, generally about 1 L in volume at a flow rate of 80 to 100 mL/sec, is guided into the microreactor 46 within the breath collection tool 10, 110, 210 and, as shown in FIG. 4B, microfluidic channels 62 direct the flow radially outward and through vent holes 58 lined at the perimeter 56 of the microreactor 46. Breath exiting the microreactor 46 flows out of the tool 10, 110, 210 via the second passageway 42, 142, 242 and outlet 16, 116, 216 through the attached filter. After completion of the breath test, the mouthpiece and filter units may be discarded into appropriate biohazardous waste containers. A disposable syringe (not shown) containing 0.5 mL methanol is then fitted onto the inlet 14, 114, 214 and a sample collection vial (not shown) is fitted onto the outlet 16, 116, 216. Using pressure from the syringe, the methanol is forced through the microreactor 46 to elute carbonyl-containing VOC adducts from the micropillars 60 and into the sample vial. The sample vial is then disconnected for use in subsequent classic GC or portable GC analysis. The breath collection tool 10, 110, 210 with attached syringe may then be discarded into a biohazardous waste container.

Release of the captured target compounds can be enacted by a variety of methods apart from elution via methanol injected into the inlet 14, 114, 214. In other embodiments, depending upon the capture material and target compounds captured by the capture material, a different eluant other than methanol may be used. In further embodiments (not shown), the breath collection tool may include an opening into the first passageway additional to the inlet, such as an opening on the side of the upper section, the additional opening used for introducing eluant into the first passageway. In another option (not shown), eluant may introduced into the optional mouthpiece 221 using the same opening that receives exhaled breath or a separate opening specific to the eluant. In some embodiments, instead of forcing eluant into the breath collection tool 10, 110, 210, a vacuum may be attached downstream of the outlet 16, 116, 216 and used to draw the eluant through the breath collection tool 10, 110, 210. In alternative embodiments, the breath collection tool 10, 110, 210 may be placed in a centrifuge or otherwise rotated and centripetal force used to draw eluant through the tool 10, 110, 210. Other options include removing the microreactor 46 from the breath collection tool 10, 110, 210 prior to eluting the target compound or for storage of the target compound. Once removed, from the tool 10, 110, 210, eluant may be introduced into the entry port 64 and removed via the vent holes 58 or introduced into the vent holes 58 and removed via the entry port 64, or the microreactor 64 may be placed in an eluant bath. Another option includes removing the microreactor 46 from the breath collection tool 10, 110, 210, then releasing the captured target compound from the microreactor via gaseous removal instead of liquid elution, such as for example, by heating the microreactor 46 to about 300° C. Further options for use of the breath collection tool 10, 110, 210 include collecting an exhaled breath sample or other gaseous sample in a bag or other storage means, then attaching the storage means to the inlet 14, 114, 214 and flowing the gaseous sample from the storage means into the breath collection tool 10, 110, 210.

The disposable breath collection tool enables safe collection of a breath sample without spreading viruses from patients because of the filter and methanol elution. The microreactor with a coated reactive compound captures and concentrates carbonyl compounds from exhaled breath. The captured analytes are eluted by a small volume of methanol (100 μL to 1 mL, or about 500 μL) which, from an exhaled breath sample of about 1 L to about 5 L in volume, results in concentration up to 10,000 times. This concentration process enables a portable GC analysis for screening of COVID-19 and standard UHPLC-MS analysis for diagnosis in clinical labs. Analyses of headspace samples of COVID-19 and other virus infected cell cultures will provide a direct support for using exhaled breath analysis to detect COVID-19 positive patients and establish an effective approach for future detection of unknown virus infections.

Innovations of the present invention include, but are not limited to, (1) the structure of the breath collection tool and microreactor therein, (2) the small size and inexpensive nature of the breath collection tool, including the microreactor, allows for breath analysis via an inexpensive, one-use disposable device that avoids the need for subsequent sterilization protocols; (3) the chemoselective retention and concentration of carbonyls by the microreactor enables analysis by portable GC instruments, which have columns with fewer theoretical plates than classic GC and are otherwise overwhelmed by the large number of VOCs present in exhaled breath; (4) methanol elution of the breath collection tool not only transitions carbonyl-containing VOCs from a gaseous medium (i.e., exhaled breath) to a liquid medium (i.e., solvent) but also concentrates the carbonyl-containing VOCs and avoids spreading viruses by inactivating viruses trapped inside the microreactor; and (5) the viability of the breath collection tool as a platform technology useful for capturing a target compounds in gaseous samples, concentrating the compounds, and transitioning the compounds to a liquid medium via elution, which is suitable for use with a wide variety of target compounds by varying the capture medium within the microreactor. Accordingly, the disclosed breath collection tool can be used to facilitate rapid and portable diagnosis of multiple disease states, not only COVID-19, and may also be used for capturing target compounds unrelated to disease states.

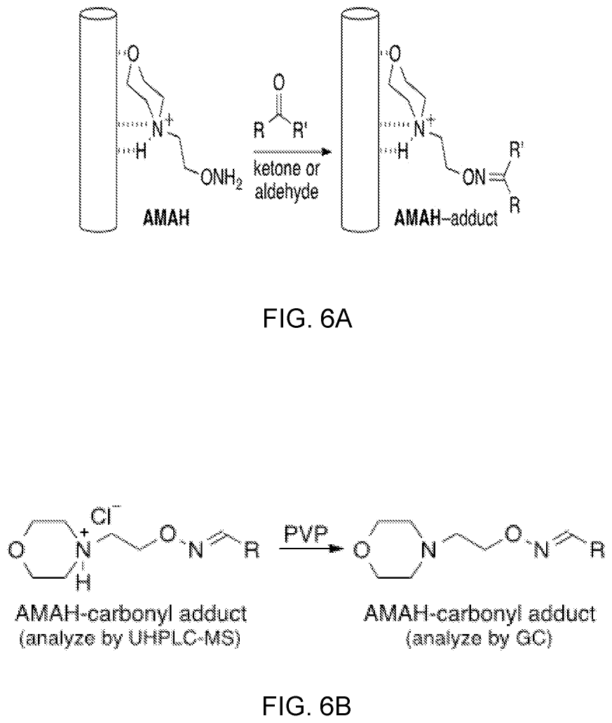

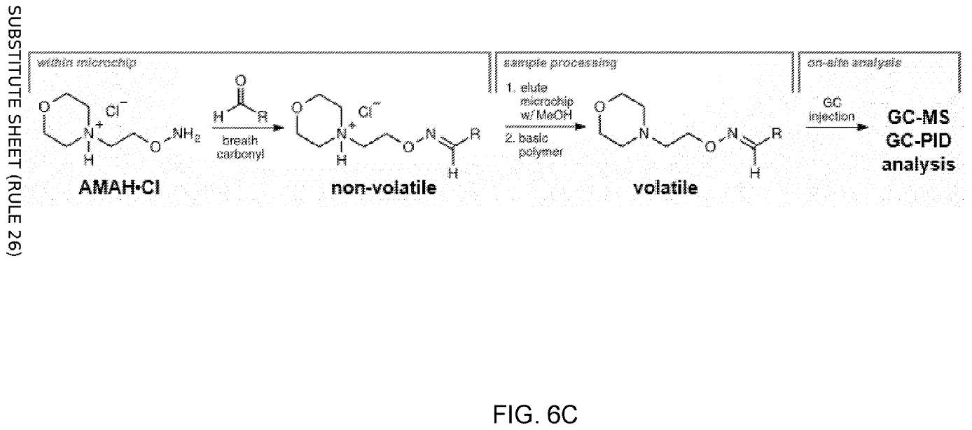

Reaction of the aminooxy (ONH₂) reactive compounds that coat the micropillars with carbonyl-containing VOCs as they pass through the microreactor converts the volatile carbonyls into non-volatile cationic (ammonium) oxime ether adducts. The cationic charge imparted to each carbonyl by derivatization facilitates analysis using mass spectrometry. Selective and fast reaction of the aminooxy moiety with carbonyl-containing VOCs is preferred for isolation of the volatile carbonyl metabolome from the complex VOC mixture in breath. In some embodiments, the aminooxy reactive compounds include a beta-ammonium hydrogen, such as is present in 4-(2-aminooxyethyl)-morpholin-4-ium chloride (AMAH⋅Cl), 2-(aminooxy)-N,N-dimethylethan-1-ammonium chloride (ADMH⋅Cl) and others, for accelerating the rate-limiting step of oxime ether formation. FIG. 6A shows a schematic illustration of a micropillar coated with AMAH as the reactive compound, and the chemical reaction between the AMAH coating and a carbonyl-containing ketone or aldehyde to capture the chemical as non-volatile adduct. An additional advantage of beta-ammonium hydrogen-containing aminooxy salts is that after carbonyl capture, the adduct can be treated with a basic resin such as poly-4-vinylpyridine (PVP) to generate the corresponding neutral adduct, which then is readily analyzed by GC-PID. FIG. 6B depicts the chemical reaction between a charged AMAH-carbonyl adduct and PVP to form a neutral AMAH-carbonyl adduct. The high activity and flexibility inherent to these beta-ammonium hydrogen-containing aminooxy salt reactive compounds is useful as the captured carbonyl-containing VOCs can readily be initially characterized using GC-MS, UHPLC-MS or portable GC. FIG. 6C summarizes the process of reacting an AMAH coating with a carbonyl-containing VOC in a breath sample within the microreactor, subsequent sample processing by eluting the adduct from the microreactor using methanol, reacting the AMAH-carbonyl adduct with a basic polymer, such as PVP, to form a volatile neutral AMAH-carbonyl adduct, and injecting the volatile adduct into a GC-MS or GC-PID for analysis. In embodiments intended to be analyzed via UHPLC-MS, elution is performed using a mixture of ethanol and water. In further embodiments, other solvents such as acetonitrile or dichloromethane may be used for elution.

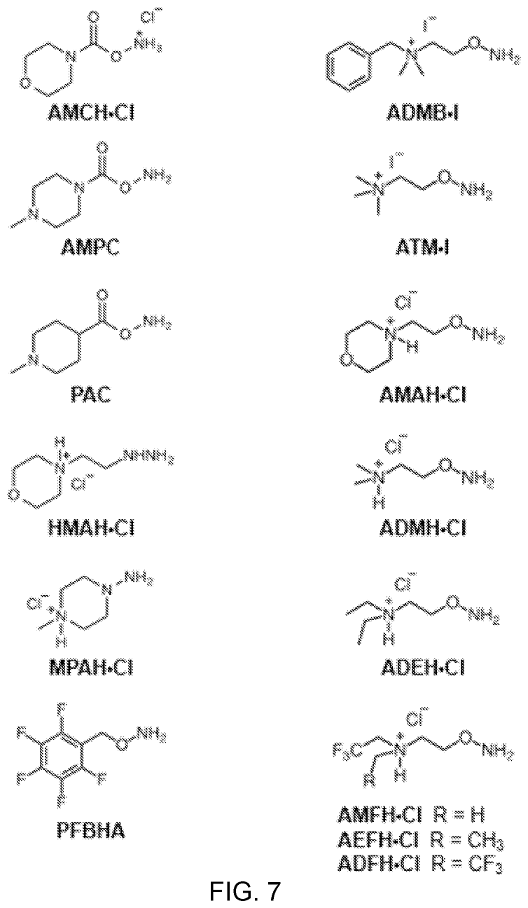

FIG. 7 displays a panel of exemplary aminooxy and hydrazine reactive compounds capable of forming adducts with carbonyl-containing VOCs. The reagents in this panel have been selected to further enhance the rate of oxime ether formation by inductive activation of the ammonium-H by incorporation of one or two electron-withdrawing CF₃ groups as well as modulation of the steric bulk surrounding the ammonium nitrogen. The ammonium salt counterion is shown as Cl⁻ or I⁻, but other counterions such as triflate (CF₃SO₃⁻) may also be used. In some embodiments, reactive compounds may be prepared as iodide salts and then reacted to provide chloride or triflate salts using ion exchange resin and AgOTf anion metathesis approaches, respectively.

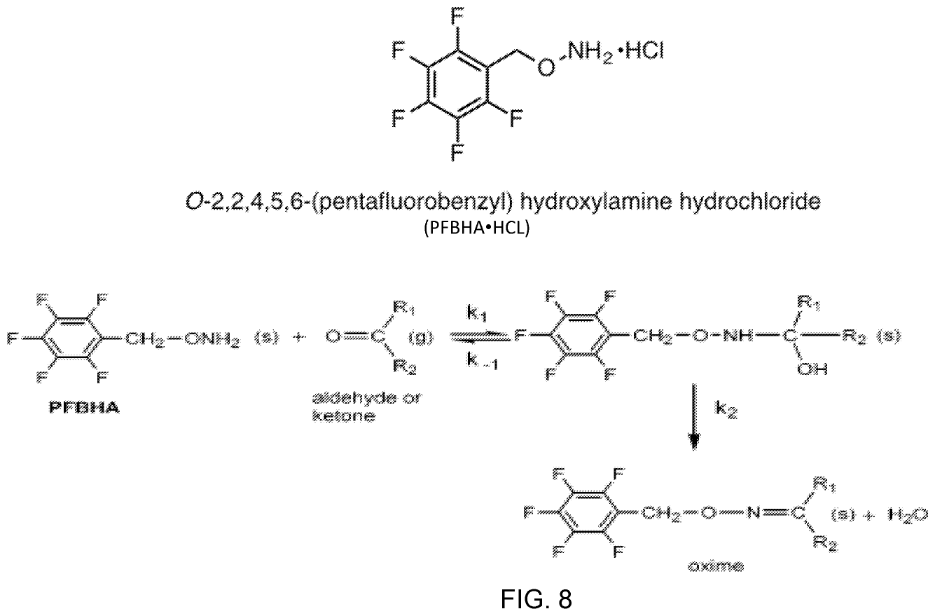

Referring to aminooxy reactive compound O-2,2,4,5,6-(pentafluorobenzyl) hydroxylamine (PFBHA), specifically, this compound has been previously used to capture aldehydes and acetone when loaded onto a polydimethylsiloxane-divinylbenzene crosslinked fiber, typical of a solid-phase microextraction (SPME) approach for carbonyl analysis. The resultant PFBHA-carbonyl adducts (oxime ethers) were analyzed by heat desorption from the fiber by direct placement of the fiber into a heated GC injection port. In the present invention, PFBHA⋅HCl is loaded onto silicia micropillars, not SPME fibers, and PFBHA-carbonyl adducts are collected via cold solvent elution, not heat desorption. FIG. 8 shows the chemical structure of PFBHA, along with the two-step reaction of PFBHA with a carbonyl-containing aldehyde or ketone to produce an oxime adduct.

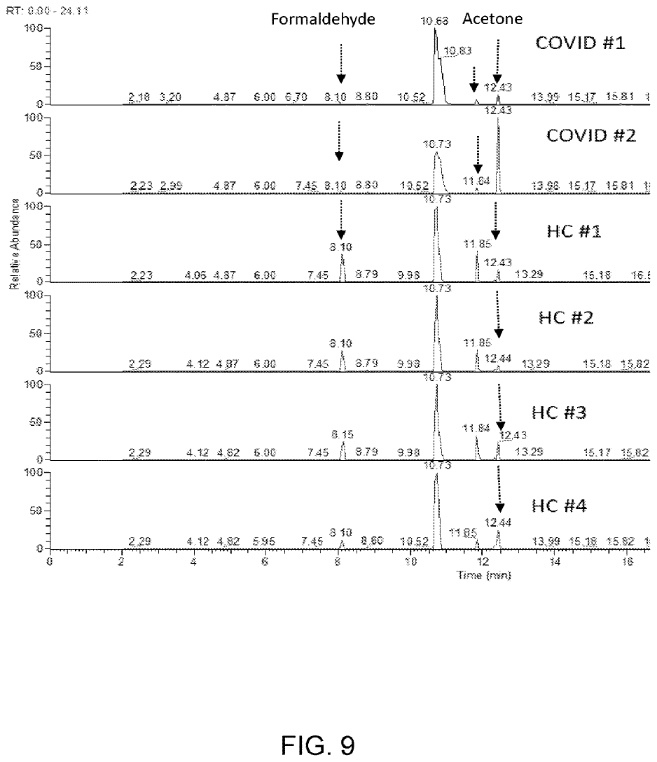

The disclosed breath collection tool has been shown effective in capturing and concentrating carbonyl-containing VOCs. Referring now to FIG. 9, a series of GC-MS chromatographs show the carbonyl-containing VOCs captured from exhaled breath using PFBHA-coated micropillars. The top two chromatographs show results from patients independently identified as having COVID-19, and the bottom four chromatographs show results from healthy control (HC) individuals. Acetone and formaldehyde are specifically identified.

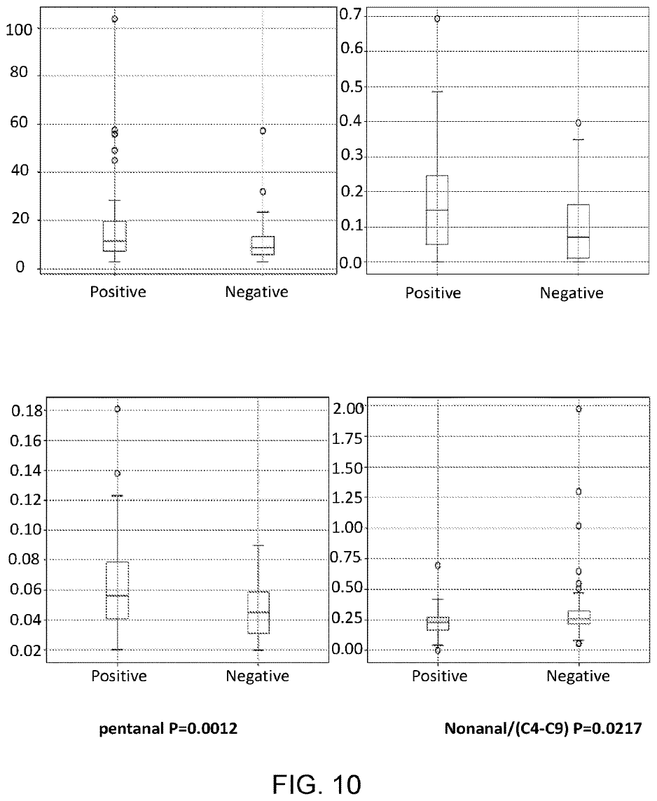

Referring now to FIG. 10, box plots present a statistical comparison between COVID-positive and -negative groups using a Wilcoxon test. The median, lower, and upper quartiles are identified. Levels of detected acetone (top left), 2-butanone (top right), pentanal (bottom left) and nonanal/(C4-C9) (bottom right) are shown, with a statistically significant difference between COVID-positive and COVID-negative subjects with a sensitivity of 0.939 and a specificity of 0.984.

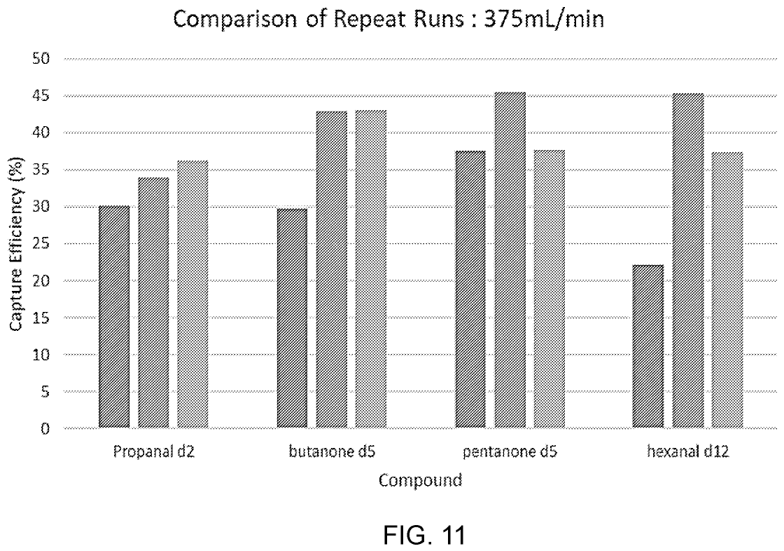

Referring now to FIG. 11, the graph compares the capture efficiency of the breath collection tool over three repeated runs of gaseous samples including propanal d2, butanone d5, pentanone d5 or hexanal d12 at a flow rate of 375 mL/min. The “d #” term following the chemical name in this figure and elsewhere in the specification refers to the number of hydrogen atoms replaced with deuterium atoms in the chemical. For example, propanal d2 is propanal with two hydrogen atoms replaced with deuterium. Using deuterated chemicals in gaseous samples allows the inventors to readily distinguish VOCs captured from the samples from VOCs present in ambient air. Similar data acquired using the same four VOCs at different flow rates (not shown) evidences that higher flow rates correlate with higher capture efficiencies using the disclosed breath collection tool.

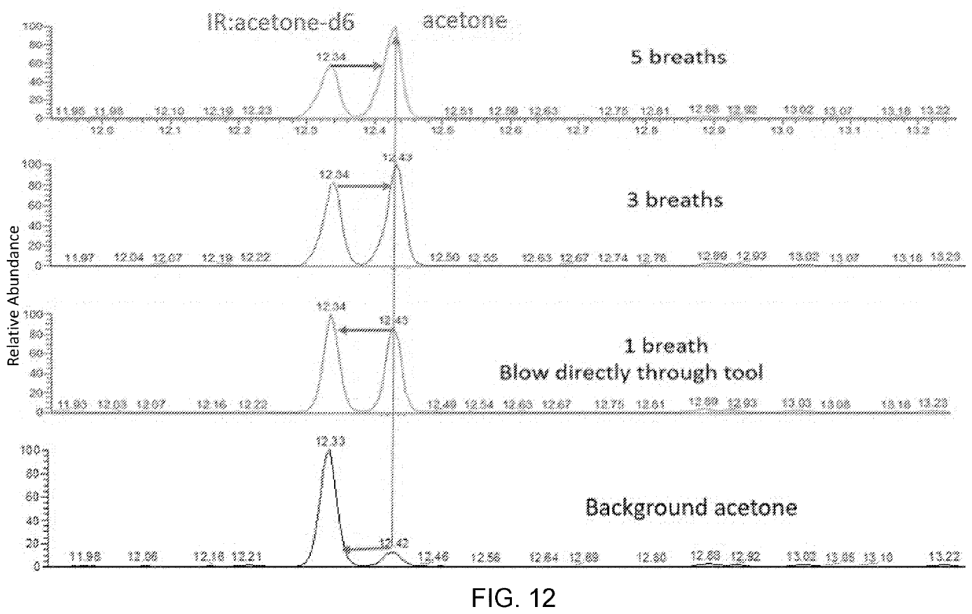

Referring now to FIG. 12, the chromatographs indicate that the amount of captured acetone increases with the number of exhaled breaths blown directly into the breath collection tool. Breath samples were collected using the disclosed breath collection tool having ATM as the capture material in the microreactor. ATM-acetone adducts were eluted from the microreactor using methanol, then the same amount of ATM-acetone-d6 was spiked into each eluted breath sample for measurement by UHPLC-MS. ATM-acetone-d6 (i.e., acetone with six hydrogen atoms replaced with deuterium atoms) serves as an internal reference for comparison with the acetone captured from the breath sample in the form of ATM-acetone adducts. FIG. 12 shows that the breath collection tool effectively captures carbonyls, in this case, acetone, and signal intensity correlates with the total volume of the breath sample. Peaks indicating the presence of acetone and acetone-d6 are labeled.

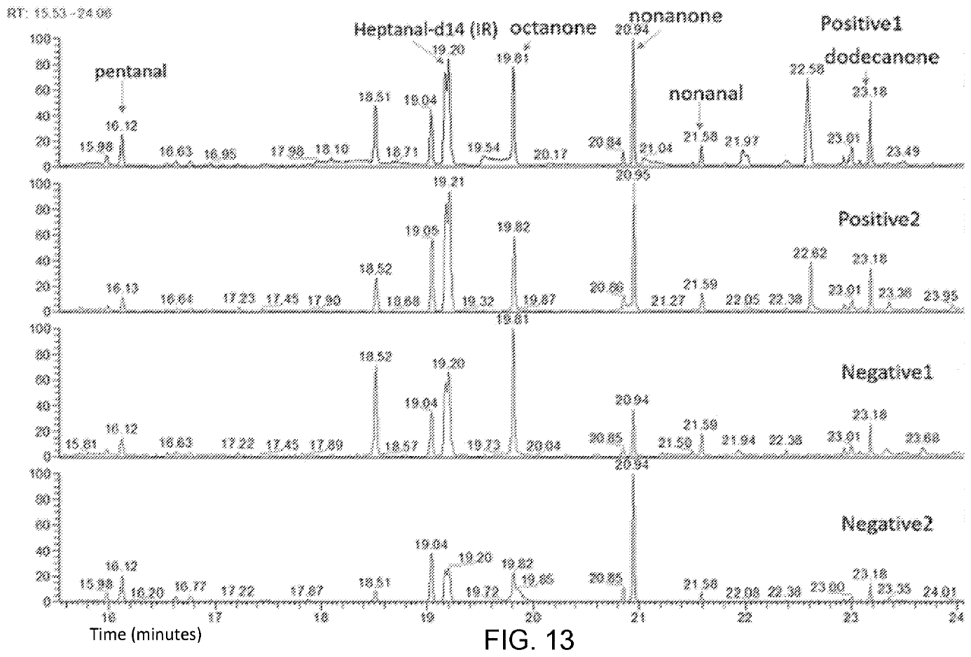

The breath collection tool is effective in capturing carbonyls of various lengths. FIG. 13 includes a series of GC-MS chromatographs showing detected carbonyls ranging from pentanal (C5) to dodecanone (C12) from exhaled breath samples captured from COVID-positive (top two chromatographs) and COVID-negative (bottom two chromatographs) subjects using the breath collection tool. Other data, not included in FIG. 13, confirms capture of shorter carbonyls, including formaldehyde (C1) and acetaldehyde (C2).

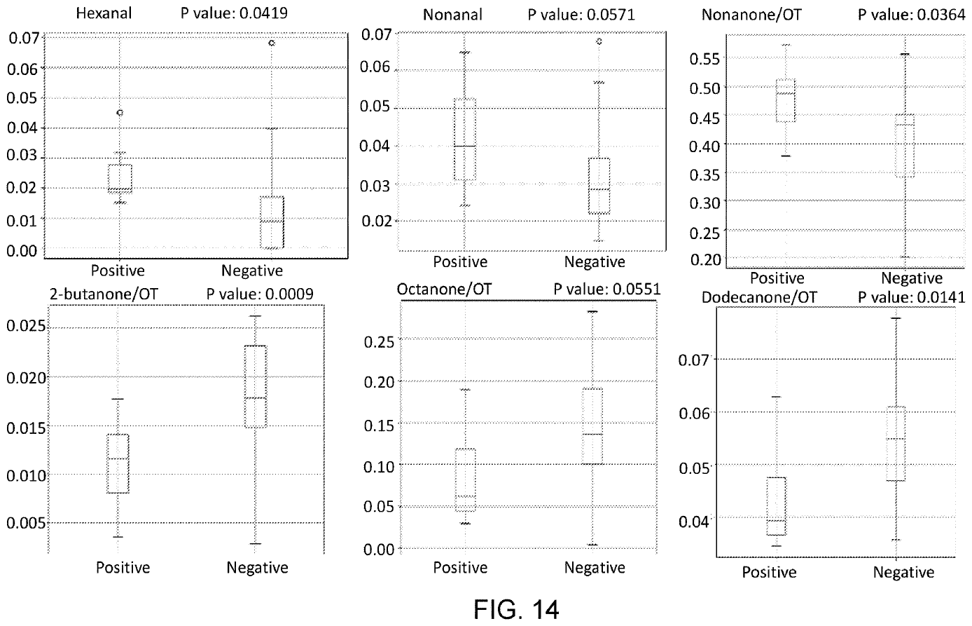

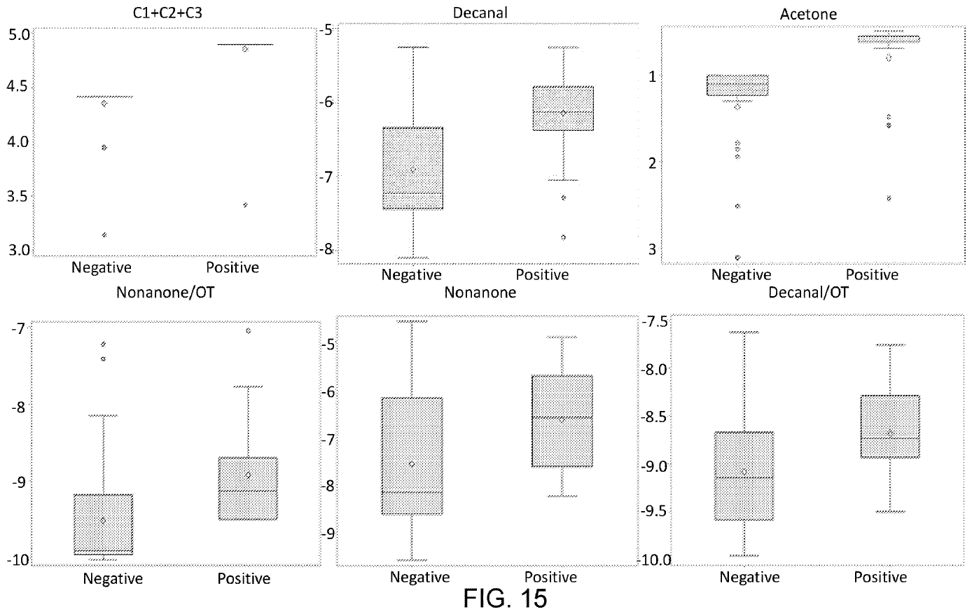

Referring now to FIG. 14, box plots present a statistical comparison between COVID-positive and COVID-negative groups using a Wilcoxon test. The median, lower, and upper quartiles are identified. Levels of detected hexanal (top left), nonanal (top middle), nonanone/OT (top right), 2-butanone/OT (bottom left), octanone/OT (bottom middle) and dodecanone/OT (bottom right) are shown, with a statistically significant difference between COVID-positive and COVID-negative subjects. The term “OT,” as used herein, is an abbreviation of “other total” and references the total amount of detected VOCs excluding acetone (propanone), acetaldehyde (ethanal) and formaldehyde (methanal), each of which are naturally present in substantial amounts. For example, the top right box plot indicates the ratio of detected nonanone to all other detected VOCs, apart from acetone, acetaldehyde and formaldehyde. As discussed later, the inventors found that ratios of detected carbonyl-containing VOCs can serve as biomarkers of COVID as effectively or more effectively than individual carbonyl-containing VOCs.

Exhaled breath samples were collected from 141 COVID-19 positive (55 alpha, 86 delta) and 181 COVID-negative subjects and analyzed by UHPLC-MS. Logistic regression was used to model the efficacy of individual biomarkers as indicative of the presence or absence of COVID-19 in a breath sample. The resulting data, as summarized in Table 1, indicates that certain carbonyl-containing VOCs or ratios of carbonyl-containing VOCs detected in the samples are effective biomarkers of the presence or absence of COVID-19.

TABLE 1
Univariable Logistic Model Results

| Compound or | | | | | | | | |
|---|---|---|---|---|---|---|---|---|
| Ratio | pv | FDR | AUC | Sensitivity | Specificity | PPV | NPV | Accuracy |
| C1 + C2 + C3 | <0.001 | <0.001 | 0.971 | 0.971 | 1.000 | 1.000 | 0.976 | 0.986 |
| Decanal | <0.001 | 0.002 | 0.785 | 0.735 | 0.775 | 0.735 | 0.775 | 0.757 |
| Acetone | <0.001 | 0.008 | 0.833 | 0.794 | 1.000 | 1.000 | 0.851 | 0.905 |
| Nonanone/ | 0.002 | 0.017 | 0.744 | 1.000 | 0.625 | 0.694 | 1.000 | 0.797 |
| OT | | | | | | | | |
| Nonanone | 0.005 | 0.037 | 0.697 | 0.882 | 0.550 | 0.625 | 0.846 | 0.703 |
| Decanal/OT | 0.007 | 0.037 | 0.669 | 0.824 | 0.600 | 0.636 | 0.800 | 0.703 |
| Nonanal/OT | 0.007 | 0.037 | 0.685 | 0.853 | 0.550 | 0.617 | 0.815 | 0.689 |
| C1/(C1 + C2 + | 0.011 | 0.052 | 0.648 | 0.412 | 0.925 | 0.824 | 0.649 | 0.689 |
| C3) | | | | | | | | |
| Dodecanone/ | 0.016 | 0.065 | 0.720 | 0.853 | 0.675 | 0.690 | 0.844 | 0.757 |
| OT | | | | | | | | |
| C3/C1 | 0.073 | 0.270 | 0.654 | 0.382 | 1.000 | 1.000 | 0.656 | 0.716 |
| Nonanal | 0.082 | 0.274 | 0.607 | 0.853 | 0.450 | 0.569 | 0.783 | 0.635 |

pv = p-value; FDR = False Discovery Rate; AUC = Area Under Curve; PPV = Positive Prediction Value; NPV = Negative Prediction Value

Referring now to FIG. 15, box plots present a statistical comparison of identified target compounds or ratios of compounds as between COVID-positive and COVID-negative groups using a Wilcoxon test. The median, lower, and upper quartiles are identified. Levels of detected C1+C2+C3 (i.e., the sum total of detected 1, 2, and 3 carbon VOCs, top left), decanal (top middle), acetone (top right), nonanone/OT (bottom left), nonanone (bottom middle) and decanal/OT (bottom right) are shown, with a statistically significant difference between COVID-positive and COVID-negative subjects. This data corresponds with the top six entries in Table 1 above.

Using the above data, the inventors developed a machine learning-derived logistic regression model using target compounds, combinations of target compounds, or ratios of target compounds as parameters for categorizing a sample as indicative of the presence or absence of COVID:

$$Log [ ( {p}^{*} / 1 - {p}^{*} ) ] = {α}^{* \hat{}} + β {1}^{* \hat{}} C 10 + … + β {k}^{* \hat{}} Ck 0 \qquad ( Eq 1 )$$

If p*>0.50, the sample is classified as COVID-positive, otherwise the sample is classified as COVID-negative. The logistic regression model was generated based on a training set of 60 COVID omicron positive samples and 40 COVID negative samples, then tested on a training set of 24 COVID omicron positive samples and 20 COVID negative samples. The logistic regression model was based off 7 parameters, namely, the top 7 target compounds, combinations of target compounds, or ratios of target compounds identified in Table 1 above. The model includes the following preprocessing steps: (i) Min-Max Scaler, (ii) Polynomial Features and (iii) Set Percentile. The model includes two classifiers: (i) Multi-Layer Perception (MLP/NN) and (ii) Decision Tree. From testing, the model was determined to have an accuracy of 93.18%, a sensitivity of 95.83%, a specificity of 90.00%, and a precision of 92.00%.

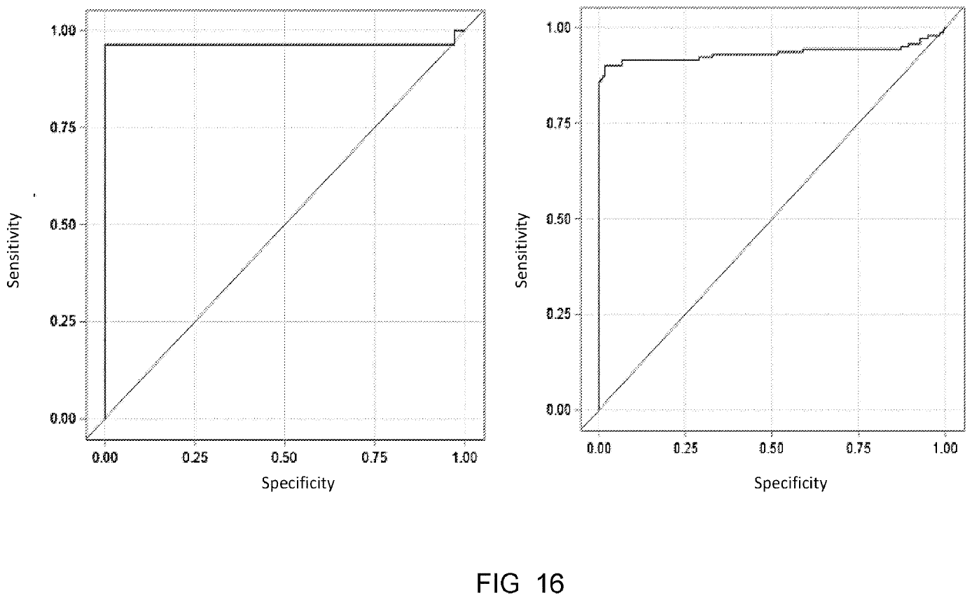

FIG. 16 includes a pair of graphs depicting the results of machine learning algorithms for detection of positives of 55 alpha, 86 delta and 181 negatives by UHPLC-MS analysis of exhaled breath samples collected using the disclosed breath collection tool. The left graph depicts the ROC curve for analysis of exhaled breath samples by measurement of the acetone/butanone ratio and pentanal/OT ratio. The AUC is 0.9647. The right graph depicts the ROC curve for analysis of exhaled breath samples by measurement of acetone, butanal/OT, (C1+C2+C3), hexanal, and acetone/butanone. The AUC is 0.9327.

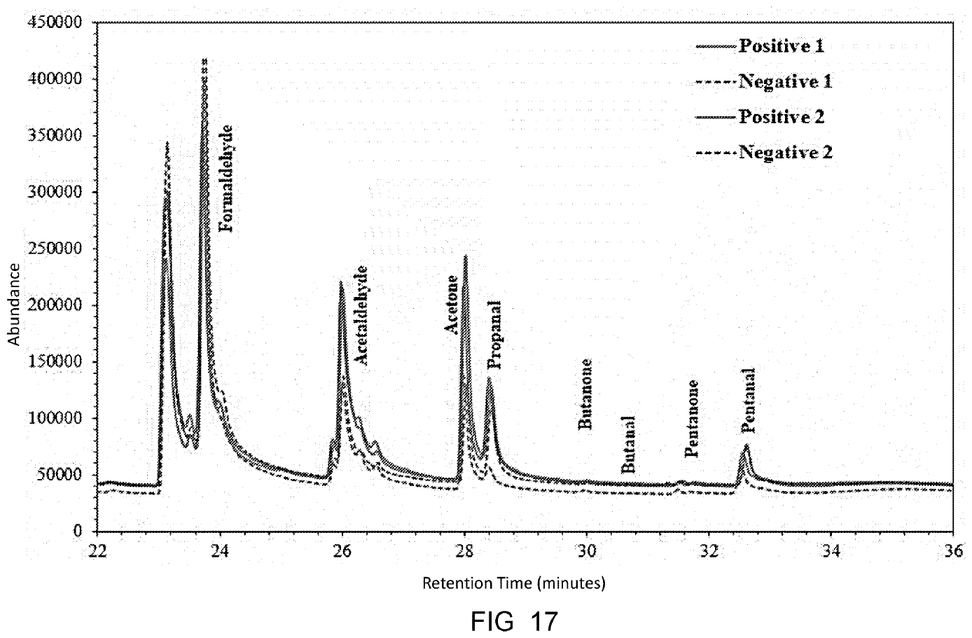

FIG. 17 displays GC-PID chromatographs of exhaled breath samples of COVID-19 positive (solid lines) and COVID-19 negative (dashed lines) captured using the breath collection tool and analyzed by a portable GC-PID.

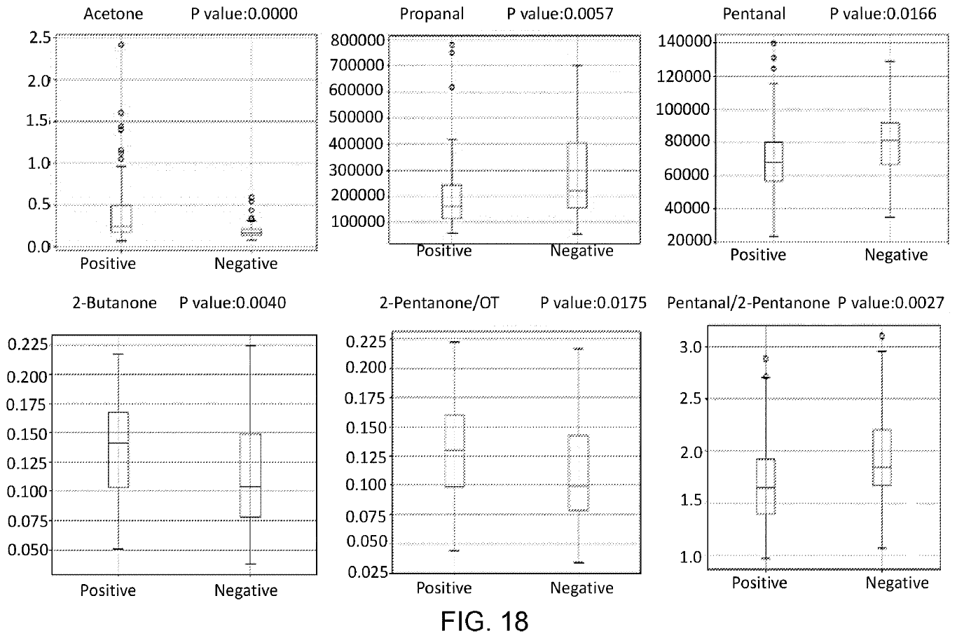

Referring now to FIG. 18, box plots present a statistical comparison between COVID-positive and COVID-negative groups using a Wilcoxon test. The median, lower, and upper quartiles are identified. Levels of detected acetone (top left), propanal (top middle), pentanal (top right), 2-butanone (bottom left), 2-pentanone/OT (bottom middle) and pentanal/pentanone (bottom right) are shown, with a statistically significant difference between COVID-positive and COVID-negative subjects. Breath samples were captured via the disclosed breath collection tool and the carbonyl content analyzed via portable GC-PID. FIGS. 17 and 18 collectively show the viability of using the disclosed breath collection tool in combination with a portable GC-PID for portable collection and analysis of exhaled breath samples.

Collectively, the inventors have shown the combination of capturing exhaled breath samples via the disclosed breath collection tool, analyzing the sample via portable GC-PID, and categorization of the sample by the disclosed model provides an effective, portable means for COVID-19 screening.

The foregoing detailed description is given primarily for clearness of understanding and no unnecessary limitations are to be understood therefrom for modifications can be made by those skilled in the art upon reading this disclosure and may be made without departing from the spirit of the invention.

1. An apparatus for capturing one or more target compounds in a gaseous sample, the apparatus comprising:
a microreactor including a first side and a second side opposite the first side,
a circular recess formed in the first side, the circular recess including a central area and a perimeter,
a plurality of vent holes located within the recess, the vent holes spaced along the perimeter and extending through the microreactor, and
a plurality of micropillars positioned within the recess between the central area and the perimeter, the plurality of micropillars forming a plurality of microfluidic channels between the central area and the perimeter;
wherein the plurality of micropillars are coated with a capture material capable of capturing the one or more target compounds.

2. The apparatus of claim 1, further comprising an apparatus body including a hollow interior, an inlet and an outlet; wherein the microreactor is located within the interior in fluid communication with the inlet and outlet, such that airflow into the inlet is directed toward the central area of the first side, is redirected radially through the plurality of microfluidic channels, through the plurality of vent holes, and exits the body through the outlet.

3. The apparatus of claim 2,
wherein the apparatus body includes an upper section and a lower section, the upper section and lower section being removably attachable to each other;
wherein the upper section includes
an upper body having an upper end including the inlet, a lower end, at least one side between the upper end and the lower end, and
a first passageway extending from the inlet, through the upper body, and terminating in a nozzle opposite the inlet;
wherein the lower section includes
a lower body having an upper end, a lower end, at least one side between the upper end and the lower end, and a tube extending from the lower end and terminating in the outlet, and
a second passageway extending from the upper end, through the lower body, and terminating in the outlet;
wherein the nozzle is inserted at least partially within the upper end of the lower body; and
wherein the first passageway and second passageway cooperatively form the hollow interior of the apparatus body.

4. The apparatus of claim 3, further comprising a tube extending from the upper end of the upper body, wherein the tube terminates in the inlet.

5. The apparatus of claim 3, wherein the microreactor is positioned within a cavity in the second passageway sized and shaped to receive the microreactor between the nozzle and the outlet.

6. The apparatus of claim 3, further comprising a cover attached to the first side of the microreactor, the cover including an entry port aligned over the central area, wherein the circular recess and cover cooperatively define an interior of the microreactor, such that airflow into the inlet sequentially passes through the first passageway, nozzle, entry port, interior of the microreactor, vent holes of the microreactor, second passageway, and outlet.

7. The apparatus of claim 6, wherein the nozzle engages and is received by the entry port of the cover.

8. The apparatus of claim 1, further comprising a cover attached to the first side of the microreactor, the cover including an entry port aligned over the central area, wherein the circular recess and cover cooperatively define an interior of the microreactor.

9. The apparatus of claim 1, wherein the plurality of micropillars includes micropillars of different diameters, wherein micropillars with larger diameters are located closer to the central area than micropillars with smaller diameters, and wherein micropillars with smaller diameters are located closer to the perimeter than micropillars with larger diameters.

10. The apparatus of claim 1, wherein spacing between micropillars in the plurality of micropillars varies, with greater spacing between micropillars in proximity to the central area than between micropillars in proximity to the perimeter.

11. The apparatus of claim 1, wherein micropillars in the plurality of micropillars are arranged such that radial airflow from the central area to any of the plurality of vent holes is deflected around multiple micropillars.

12. The apparatus of claim 1, further comprising an apparatus body including a hollow interior, an inlet and an outlet; wherein the microreactor is located within the interior in fluid communication with the inlet and outlet, such that airflow into the inlet is directed toward the central area of the first side, is redirected radially through the plurality of microfluidic channels, through the plurality of vent holes, and exits the body through the outlet.

13. An apparatus for capturing one or more target compounds in a gaseous sample, the apparatus comprising:
an apparatus body including an upper section and a lower section, the upper section and lower section being removably attachable to each other;
wherein the upper section includes
an upper body having an upper end including an inlet, a lower end, at least one side between the upper end and the lower end, and
a first passageway extending from the inlet, through the upper body, and terminating in a nozzle opposite the inlet;
wherein the lower section includes
a lower body having an upper end, a lower end, at least one side between the upper end and the lower end, and a tube extending from the lower end and terminating in an outlet, and
a second passageway extending from the upper end, through the lower body, and terminating in the outlet;
wherein the nozzle is inserted at least partially within the upper end of the lower body; and
wherein the first passageway and second passageway cooperatively form a hollow interior of the apparatus body; and
a microreactor within the second passageway in fluid communication with the inlet and outlet, such that airflow into the inlet sequentially passes through the first passageway, nozzle, microreactor, second passageway, and outlet.

14. The apparatus of claim 13, further comprising a tube extending from the upper end of the upper body, wherein the tube terminates in the inlet.

15. The apparatus of claim 13, wherein the microreactor is positioned within a cavity in the second passageway sized and shaped to receive the microreactor between the nozzle and the outlet.

16. The apparatus of claim 13,
wherein microreactor includes a first side and a second side opposite the first side,
a circular recess formed in the first side, the circular recess including a central area and a perimeter,
a plurality of vent holes located within the recess, the vent holes spaced along the perimeter and extending through the microreactor, and
a plurality of micropillars positioned within the recess between the central area and the perimeter, the plurality of micropillars forming a plurality of microfluidic channels between the central area and the perimeter;
wherein the plurality of micropillars are coated with a capture material capable of capturing the one or more target compounds.

17. The apparatus of claim 16, further comprising a cover attached to the first side of the microreactor, the cover including an entry port aligned over the central area, wherein the circular recess and cover cooperatively define an interior of the microreactor, such that airflow into the inlet sequentially passes through the first passageway, nozzle, entry port, interior of the microreactor, second passageway, and outlet.

18. The apparatus of claim 17, wherein the nozzle engages and is received by the entry port of the cover.

19. A method of capturing one or more target compounds in a gaseous sample, the method comprising:
providing an apparatus according to claim 1;
directing an airflow of the gaseous sample toward the central area, such that the airflow contacts the central area and is redirected through the plurality of microfluidic channels formed by the plurality of micropillars and into the vent holes; and
capturing the one or more target compounds using the capture material on the plurality of micropillars.

20. The method of claim 19, further comprising eluting the one or more target compounds from the plurality of micropillars using an eluant.

21. The method of claim 20, wherein the eluant is methanol.

22. The method of claim 20, further comprising, after said eluting, determining a binary outcome based on the captured one or more target compounds.

23. The method of claim 22, wherein the gaseous sample is an exhaled breath sample, wherein the one or more target compounds are carbonyl-containing volatile organic compounds, and wherein the binary outcome is the presence or absence of a disease state in the provider of the exhaled breath sample.

24. The method of claim 20, wherein the apparatus further comprises a cover attached to the first side of the microreactor, the cover including an entry port aligned over the central area, and wherein the circular recess and cover cooperatively define an interior of the microreactor; and
wherein said eluting includes introducing the eluant into the interior of the microreactor through the entry port.

25. The method of claim 19, wherein the capture material is a reactive compound, and wherein the capturing comprises forming adducts between the reactive compound and the one or more target compounds.

26. The method of claim 19, wherein the capture material is an adsorptive material.

27. The method of claim 19, wherein the one or more target compounds are carbonyl-containing volatile organic compounds.

28. The method of claim 19, wherein the gaseous sample is an exhaled breath sample.

29. The method of claim 19, further comprising determining a binary outcome based on the captured one or more target compounds.

30. The method of claim 29, wherein the gaseous sample is an exhaled breath sample and wherein the binary outcome is the presence or absence of a disease state in the provider of the exhaled breath sample.

31. The method of claim 30, wherein the disease state is COVID-19.

32. The method of claim 29, wherein the determining is performed using a machine learning system based on concentrations or ratios of concentrations of the captured one or more target compounds as input parameters to the machined learning system.

33. A method of capturing one or more target compounds in a gaseous sample, the method comprising:
providing an apparatus according to claim 13;
directing an airflow of the gaseous sample into the inlet and sequentially through the first passageway, nozzle, microreactor, second passageway, and outlet; and
capturing the one or more target compounds using a capture material within the microreactor.

34. The method of claim 33, further comprising eluting the one or more target compounds from the microreactor using an eluant.

35. The method of claim 34, wherein the eluant is methanol.

36. The method of claim 34, wherein the eluant is introduced into the apparatus through the inlet and exits the apparatus through the outlet.

37. The method of claim 34, wherein the eluant is introduced into the microreactor without removing the microreactor from the apparatus.

38. The method of claim 34, wherein the eluant is introduced into the microreactor after removing the microreactor from the apparatus.

39. The method of claim 33, wherein the capture material is a reactive compound, and wherein the capturing comprises forming adducts between the reactive compound and the one or more target compounds.

40. The method of claim 33, wherein the capture material is an adsorptive material.

41. The method of claim 33, wherein the one or more target compounds are carbonyl-containing volatile organic compounds.

42. The method of claim 33, wherein the gaseous sample is an exhaled breath sample.

43. The method of claim 33, further comprising determining a binary outcome based on the captured one or more target compounds.

44. The method of claim 43, wherein the gaseous sample is an exhaled breath sample and wherein the binary outcome is the presence or absence of a disease state in the provider of the exhaled breath sample.

45. The method of claim 44, wherein the disease state is COVID-19.

46. The method of claim 43, wherein the determining is performed using a machine learning system based on concentrations or ratios of concentrations of the captured one or more target compounds as input parameters to the machined learning system.
